(19) United States
(12) Patent Application Publication
(10) Pub. No.: US 2025/0048924 A1
(43) Pub. Date: Feb. 6, 2025

(54) ORGANIC COMPOSITION AND ORGANIC OPTOELECTRONIC DEVICE USING THE SAME

(71) Applicant: Raynergy Tek Incorporation, Hsinchu (TW)

(72) Inventors: Yu-Tang Hsiao, Hsinchu (TW); Chuang-Yi Liao, Hsinchu (TW)

(21) Appl. No.: 18/792,053

(22) Filed: Aug. 1, 2024

Related U.S. Application Data
(60) Provisional application No. 63/530,709, filed on Aug. 4, 2023.

(51) Int. Cl. H10K 85/60; C07D 495/22; C07D 513/22; C07D 517/22; H10K 50/11; H10K 85/10; H10K 85/20; H10K 85/30

(52) U.S. Cl. CPC H10K 85/657; C07D 495/22; C07D 513/22; C07D 517/22; H10K 85/111; H10K 85/211; H10K 85/30; H10K 85/626; H10K 85/655; H10K 50/11

(57) ABSTRACT

An organic composition comprises at least one donor material and at least one acceptor material. The donor material comprises at least one organic conjugated polymer or organic conjugated small molecule. The acceptor material comprises a first acceptor material, and the first acceptor material comprises Formula I:

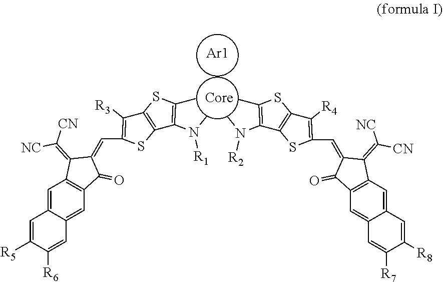

The naphthalene ring group at the end of the Formula I can effectively improve the stability of the material. Substituents R₅, R₆, R₇ and R₈ with at least one substituent can adjust the energy level of the material to facilitate matching with different donor materials. The invention also provides an organic optoelectronic device comprising a first electrode, an active layer and a second electrode. The active layer comprises the organic composition. The organic optoelectronic device has good responsibility, dark current, detectivity and photoelectric conversion efficiency, and the organic optoelectronic device has good stability.

The present application is based on, and claims priority from, America provisional patent application number U.S. 63/530,709, filed on 2023 Aug. 4, and the disclosure of which is hereby incorporated by reference herein in its entirety.

### BACKGROUND OF THE INVENTION

### Field of the Invention

The present invention relates to an organic composition applied to an organic optoelectronic device, and in particular to an organic composition containing a donor material and an acceptor material, and an organic optoelectronic device using the same.

### Description of the Prior Art

Compared to traditional inorganic optoelectronic devices, organic optoelectronic devices have a wide absorption wavelength range, high absorption coefficient, and adjustable structures, and their light absorption range, energy level and solubility can be adjusted according to the target requirements. In addition, organic materials have the advantages of low cost, flexibility, low toxicity and large-area production of devices, so that organic optoelectronic devices have good competitiveness in various fields, such as organic field effect transistors (OFETs), organic light emitting diodes (OLEDs), organic photovoltaics (OPVs) and organic photodetectors (OPDs).

The organic photodetector will have different material requirements according to different applications, and the application requirements for invisible region will be greatly increased. For example, biometric technologies, such as vein scanners, iris sensors, and facial recognition, as well as the physiological vital sign monitoring technology of pulse oximetry measurement, whose demand has increased due to the COVID-19 epidemic, and machine vision applications like LiDAR and time of flight sensors that are currently required by self-driving cars. Therefore, how to provide an organic photodetector with high performance and low cost in the absorption range of near infrared or shortwave infrared corresponding to the above applications is a very important issue at present.

Active layer materials play an important role in organic photodetectors and will directly affect device performance. The active layer material is divided into two parts: donor materials and acceptor materials. For the donor materials, the development of D-A conjugated polymers is the mainstream. The electron push-pull effect of electron-rich units and electron-deficient units in conjugated polymers can be used to control the energy levels and energy band gaps of polymers. The acceptor materials blended with the donor materials are usually fullerene derivatives with high conductivity, and its light absorption range is about 400˜600 nm. However, the structure of fullerene derivatives is not easy to adjust, and their light absorption and energy levels are limited within a certain range, which limits the overall combination of the donor materials and the acceptor materials. With the development of the market, the demand for materials in the near-infrared region is gradually increasing.

Even if the light absorption range of the conjugated polymer of the donor materials can be adjusted to the near-infrared region, it may not be able to have a good match due to the limitation of fullerene acceptor materials. Therefore, it is very important to develop non-fullerene acceptor materials to replace traditional fullerene acceptor materials in the breakthrough of active layer materials. The material development of the non-fullerene acceptor materials is mainly to form A-D-A ladder type molecules with electron-rich centers and electron-deficient units on both sides. D is usually a ladder type molecule composed of benzene and thiophene, and A is usually an indanone-cyano derivative. ITIC is a representative non-fullerene acceptor with an absorption range about 600˜750 nm, and it also has good performance in organic photodetectors. In addition to the A-D-A ladder molecules, in 2019, the ladder type molecules published by Yang et. al with the A-D-A′-D-A structure, such as Y6, have a light absorption range of 600˜900 nm, which further extends the light absorption spectrum of non-fullerene acceptors to near-infrared region. Therefore, how to develop an organic composition that is a light-absorbing material covering near-infrared light and has good device performance is currently a very important issue.

### SUMMARY OF THE INVENTION

In view of this, one category of the present invention is to provide an organic composition, which has good optical properties and suitable energy levels, and can be configured as an organic optoelectronic device with a suitable p-type material.

According to a specific embodiment of the present invention, the organic composition comprises at least one donor material and at least one acceptor material.

The donor material comprises at least one organic conjugated polymer or organic conjugated small molecule. The acceptor material comprises a first acceptor material comprising Formula I:

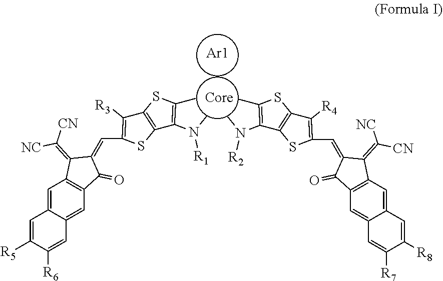

Wherein, core is phenyl. Ar1 is a five-membered or six-membered heterocyclic ring comprising at least one heteroatom, and the heteroatom is independently selected from at least one of S, N, O and Se. R₁, R₂, R₃ and R₄ are independently selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkylaryl, C1-C30 haloalkyl heteroaryl, C2-C30 esteryl aryl and C2-C30 esteryl heteroaryl. R₅, R₆, R₇ and R₈ are independently selected from the following groups and their derivatives consisting of C1-C30 alkyl, C1-C30 silyl, C1-C30 alkoxy, C1-C30 alkylthio, C1-C30 haloalkyl, halogen, hydrogen and cyano, wherein R₅, R₆, R₇ and R₈ are not hydrogen at the same time.

Wherein, Ar1 is selected from the following structures:

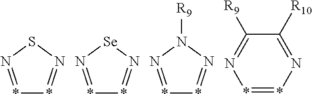

Wherein, R₉ and R₁₀ are independently selected from the following groups and their derivatives consisting of halogen, hydrogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl, wherein R₉ and R₁₀ are two separate groups or connected to each other by a single bond.

Wherein, the donor material is further selected from at least one organic conjugated polymer.

Wherein, the organic conjugated polymer is selected from the following structures:

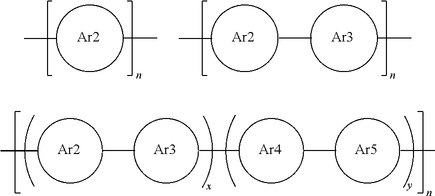

Wherein, Ar2, Ar3, Ar4 and Ar5 are monocyclic or polycyclic structures containing C4-C30 ring atoms respectively. n is the number of molecules, and n is a positive integer from 1 to 1000. x and y are molar fractions, where 0<x<1, 0<y<1 and x+y=1.

Wherein, Ar2 and Ar4 are independently selected from the following structures:

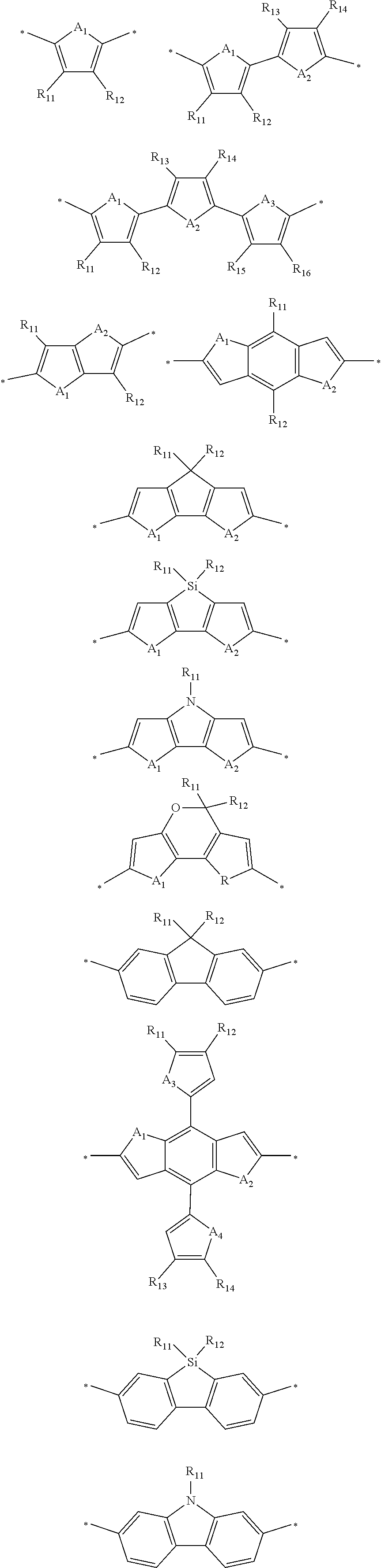

Wherein, A₁, A₂, A₃ and A₄ are independently selected from the following group consisting of O, S, Se and N—R, and R is selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl. R₁₁, R₁₂, R₁₃, R₁₄, R₁₅ and R₁₆ are independently selected from the following groups and their derivatives consisting of hydrogen, halogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkylaryl, C1-C30 haloalkyl heteroaryl, C1-C30 esteryl aryl and C2-C30 esteryl heteroaryl.

Wherein, Ar3 and Ar5 are independently selected from the following structures:

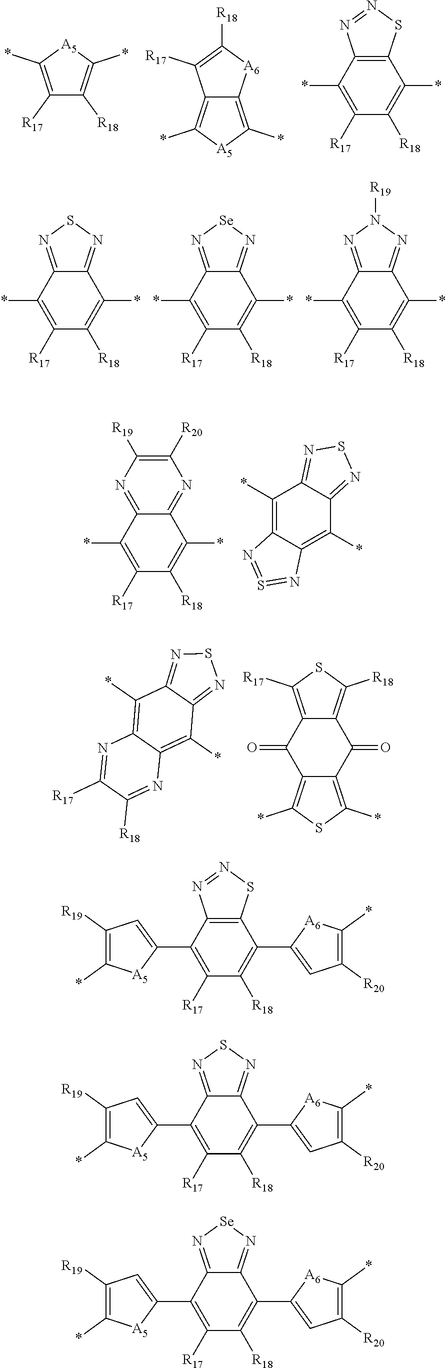

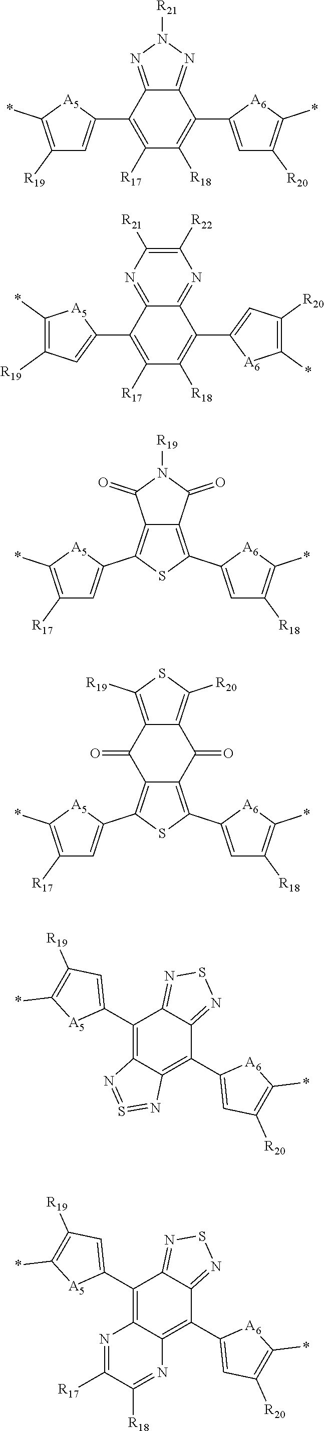

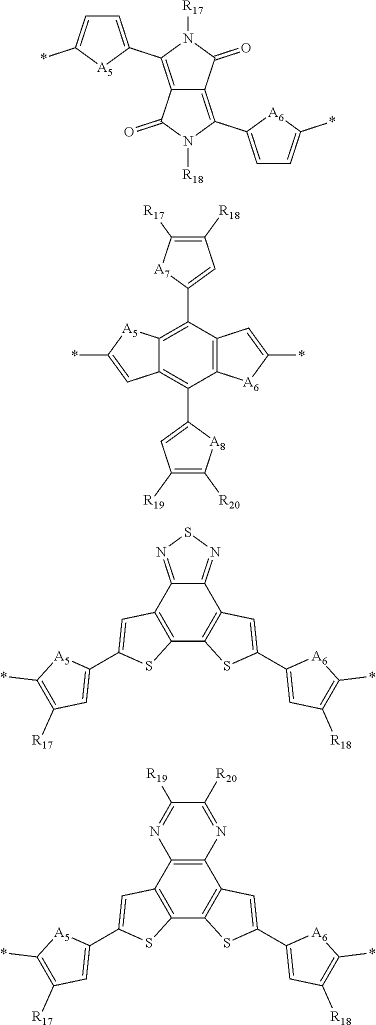

Wherein, A₅, A₆, A₇ and A₈ are independently selected from the following group consisting of O, S, Se and N—R, and R is selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl-, C1-C30 alkyl heteroaryl, C1-C30 silyl aryl, C1-C30 silyl heteroaryl, C1-C30 alkoxy aryl, C1-C30 alkoxy heteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl. R₁₇, R₁₈, R₁₉, R₂₀, R₂₁ and R₂₂ are independently selected from the following groups and their derivatives consisting of hydrogen, halogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C1-C30 esteryl aryl and C2-C30 esteryl heteroaryl.

Wherein, the acceptor material further comprises a second acceptor material, and the second acceptor material is an organic conjugated polymer or an organic conjugated small molecule.

Wherein, the second acceptor material comprises a fullerene structure.

Another category of the present invention is to provide an organic optoelectronic device comprises a first electrode, an active layer and a second electrode. The active layer comprises at least one of an organic composition aforementioned. Wherein, the active layer is disposed between the first electrode and the second electrode, and at least one of the first electrode and the second electrode is a transparent or semi-transparent electrode.

Wherein, the organic optoelectronic device further comprises a first carrier transporting layer and a second carrier transporting layer. The first carrier transporting layer is disposed between the first electrode and the active layer and the first carrier transporting layer is configured to transport carriers in the first electrode and the active layer. The second carrier transporting layer is disposed between the active layer and the second electrode and the second carrier transporting layer is configured to transport carriers in the active layer and the second electrode.

Compared with the prior art, the organic photodetector applied in the organic optoelectronic device of the present invention has good responsibility, dark current and detectivity, and the organic optoelectronic device has good stability.

### BRIEF DESCRIPTION OF THE APPENDED DRAWINGS

Some of the embodiments will be described in detail, with reference to the following figures, wherein like designations denote like members, wherein:

FIG. 1 shows a schematic structural diagram of one embodiment of an organic optoelectronic device of the present invention.

FIG. 2 shows an absorption spectra of a Comparative example 1 of the first acceptor material in thin film state and solution state.

FIG. 3 shows an absorption spectra of an embodiment 1 of the first acceptor material of the present invention in thin film state and solution state.

FIG. 4 shows an absorption spectra of an embodiment 2 of the first acceptor material of the present invention in thin film state and solution state.

FIG. 5 shows an absorption spectra of an embodiment 3 of the first acceptor material of the present invention in thin film state and solution state.

FIG. 6 shows an absorption spectra of an embodiment 4 of the first acceptor material of the present invention in thin film state and solution state.

FIG. 7 shows an absorption spectra of an embodiment 5 of the first acceptor material of the present invention in thin film state and solution state.

FIG. 8 shows thermal stability test results for absorbance of embodiments Blend 1-1 to Blend 6-1 of organic compositions of the present invention and a Comparative example Blend C1.

FIG. 9 shows thermal stability test results for absorbance of embodiments Blend 2-2 to Blend 5-2 of organic compositions of the present invention and a Comparative example Blend C2.

FIG. 10 shows thermal stability test results for absorbance of embodiments Blend 2-3 to Blend 3-3 of organic compositions of the present invention and a Comparative example Blend C3.

FIG. 11 shows detectivity test results of organic optoelectronic devices of the present invention whose active layer respectively is embodiments Blend 4-3 to Blend 4-5 and a Comparative example Blend C4.

### DETAILED DESCRIPTION OF THE INVENTION

In order to make the advantages, spirit and features of the present invention easier and clearer, it will be detailed and discussed in the following with reference to the embodiments and the accompanying drawings. It is worth noting that the specific embodiments are merely representatives of the embodiments of the present invention, but it can be implemented in many different forms and is not limited to the embodiments described in this specification. Rather, these embodiments are provided so that this disclosure will be thorough and complete.

The terminology used in the various embodiments disclosed in the present invention is only for the purpose of describing specific embodiments, and is not intended to limit the various embodiments disclosed in the present invention. As used herein, singular forms also include plural forms unless the context clearly indicates otherwise. Unless otherwise defined, all terms (including technical and scientific terms) used in this specification have the same meanings as commonly understood by one of ordinary skill in the art to which the various embodiments disclosed herein belong. The above terms (such as those defined in commonly used dictionaries) will be interpreted as having the same meaning as the contextual meaning in the same technical field, and will not be interpreted as having an idealized or overly formal meaning, unless explicitly defined in the various embodiments disclosed herein.

In the description of this specification, the description of the reference terms “an embodiment”, “a specific embodiment” and the like means that specific features, structures, materials, or characteristics described in connection with the embodiment are included in at least one embodiment of the present invention. In this specification, the schematic expressions of the above terms do not necessarily refer to the same embodiment. Furthermore, the particular features, structures, materials, or characteristics described may be combined in any suitable manner in any one or more embodiments.

### Definition

As used herein, “donor” material and “p-type” (“P-type) material refer to a semiconductor material, such as an organic semiconductor material, having holes as a primary current or charge carrier. In some embodiments, when a p-type semiconductor material is deposited on a substrate, it can provide the hole mobility greater than about 10⁻⁵ cm²/Vs.

As used herein, “acceptor” material and “n-type” (“N-type) material refer to the semiconductor material, such as the organic semiconductor material, having electrons as the primary current or the charge carrier. In some embodiments, when an n-type semiconductor material is deposited on a substrate, it can provide the electron mobility of more than about 10⁻⁵ cm²/Vs.

The “electron-withdrawing group” refers to a group or an atom with a stronger electron-withdrawing ability than that of hydrogen, that is, it has an electron-withdrawing inductive effect. The “electron-donating group” refers to a group or an atom whose electron-donating ability is stronger than that of hydrogen, that is, it has an electron-donating induction effect. The inductive effect is the effect that the bonding electron cloud moves in a certain direction on the atomic bond due to the difference in polarity (electronegativity) of atoms or groups in the molecule.

The electron cloud tends to move towards the groups or atoms with more electronegativity.

“ [symbol] ” or “*” in the structures listed herein represents the available bonding positions of this structure, but not limited thereto.

As used herein, “mobility” refers to a speed rate of the charge carrier moving through the material under the influence of an electric field. The charge carrier is the hole (positive charge) in the p-type semiconductor material and the electron (negative charge) in the n-type semiconductor material. This parameter depends on architecture of device and can be measured by field effect component or space charge limiting current.

As used herein, “polymer” refers to a very large molecule consisting of thousands of covalently bonded atoms. Polymer is composed of many repeating units, that is, monomers (composed of one or more atoms) bonded together by covalent bonds. From the point of view of physical properties, the number of repeating units should be so large that adding some more repeating units will not significantly change the physical properties. Polymer is further divided into copolymer and homopolymer. The homopolymer is polymerized from only one type of monomer. The copolymer is polymer formed by the polymerization of two or more monomers. The copolymer is divided into alternating copolymer, random copolymer, block copolymer and graft copolymer.

As used herein, “component” (such as a thin film layer) may be considered “photoactive” if it contains one or more compounds that absorb photons to generate excitons for generating photocurrent.

As used herein, “solution proceeding” refers to a process in which a compound (e.g., a polymer), material, or composition can be used in a solution state, such as spin coating, printing (e.g., inkjet printing, gravure printing, and lithography printing), spray coating, slit coating, drop casting, dip coating, and blade coating.

As used herein, “annealing” refers to a post-deposition thermal treatment to a semi-crystalline polymer film for certain duration in the environment or under decompressed or pressurized environment. “Annealing temperature” refers to the temperature at which the polymer film or the mixed film of the polymer and other molecules can perform small-scale molecular movement and rearrangement during the annealing process. Without limitation by any particular theory, it is believed that annealing can lead to an increase in crystallinity in the polymer film and enhance the carrier mobility of the polymer film or a mixed film formed by the polymer and other molecules, and the molecules are arranged alternately to achieve the effect of independent transporting paths of effective electrons and holes.

Dark current (J_{d} or J_{dark}) as used herein, also known as no-illumination current, refers to the current flows in an optoelectronic device in the absence of light irradiation.

The responsibility (R) and the detectivity (D) as used herein are based on measuring the dark current and external quantum efficiency (EQE) of the organic photodetector, and are calculated by the following formula:

$$\begin{matrix}R ( λ ) = E Q E \frac{λ q}{hc} & D =\end{matrix} \frac{R}{\sqrt{2 {qJ}_{d}}}$$

Wherein, λ is the wavelength, q is the elementary charge (1.602×10⁻¹⁹ Coulombs), h is Planck's constant (6.626×10⁻³⁴ m² kg/s), c is the speed of light (3×10⁸ m/see), and J_{d} is the dark current.

In an embodiment, an organic composition of the present invention comprises at least one donor material and at least one acceptor material. The donor material comprises at least one organic conjugated polymer or organic conjugated small molecule. The acceptor material comprises a first acceptor material comprising Formula I:

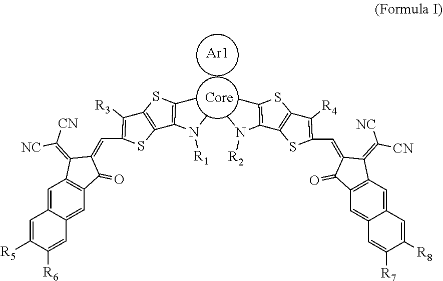

Wherein, core is phenyl. Ar1 is a five-membered or six-membered heterocyclic ring comprising at least one heteroatom, and the heteroatom is independently selected from at least one of S, N, O and Se. R₁, R₂, R₃ and R₄ are independently selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkylaryl, C1-C30 haloalkyl heteroaryl, C2-C30 esteryl aryl and C2-C30 esteryl heteroaryl. R₅, R₆, R₇ and R₈ are independently selected from the following groups and their derivatives consisting of C1-C30 alkyl, C1-C30 silyl, C1-C30 alkoxy, C1-C30 alkylthio, C1-C30 haloalkyl, halogen, hydrogen and cyano, wherein R₅, R₆, R₇ and R₈ are not hydrogen at the same time. This structure has good optical properties and appropriate energy levels and can be used as organic optoelectronic devices with appropriate p-type materials. Wherein, this structure has the following characteristics: 1. the naphthalene ring group at the end of the structure can effectively improve the stability of the material; 2. substituting R₅, R₆, R₇ and R₈ with at least one substituent can adjust the energy level of the material to facilitate matching with different p-type materials; and 3. the aforementioned substituents can also effectively change the intermolecular forces and improve the arrangement of materials, thereby improving device performance.

In practice, Ar1 is selected from the following structures:

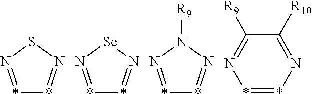

Wherein, R₉ and R₁₀ are independently selected from the following groups and their derivatives consisting of halogen, hydrogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl, wherein R₉ and R₁₀ are two separate groups or connected to each other by a single bond.

Wherein, Ar1 is further preferably selected from the following structures:

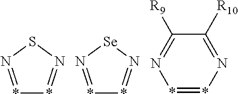

Among them, the definitions of R₉ and R₁₀ are the same as mentioned above.

In practice, R₁ and R₂ are further independently and preferably selected from C1-C30 alkyl and C1-C30 alkylaryl. R₃ and R₄ are further independently and preferably selected from the following groups and their derivatives consisting of C1-C30 alkyl, C1-C30 alkoxy, C1-C30 alkylthio, C1-C30 alkylaryl, C1-C30 alkyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, and C1-C30 haloalkyl heteroaryl. R₅, R₆, R₇ and R₈ are further independently and preferably selected from the following groups and their derivatives consisting of C1-C6 alkyl, C1-C6 alkoxy, C1-C6 haloalkyl, halogen, hydrogen and cyano, wherein R₅, R₆, R₇ and R₈ are not hydrogen at the same time.

In practice, R₁ and R₂ are independently and more preferably selected from C1-C30 alkyl. R₃ and R₄ are independently and more preferably selected from the following groups and their derivatives consisting of C1-C30 alkyl, C1-C30 alkoxy, C1-C30 alkylaryl, and C1-C30 alkyl heteroaryl. R₅, R₆, R₇ and R₈ are independently and more preferably selected from the following groups and their derivatives consisting of halogen, hydrogen and cyano, wherein R₅, R₆, R₇ and R₈ are not hydrogen at the same time.

Specifically, the first acceptor material may comprise the following embodiments 1 to 22:

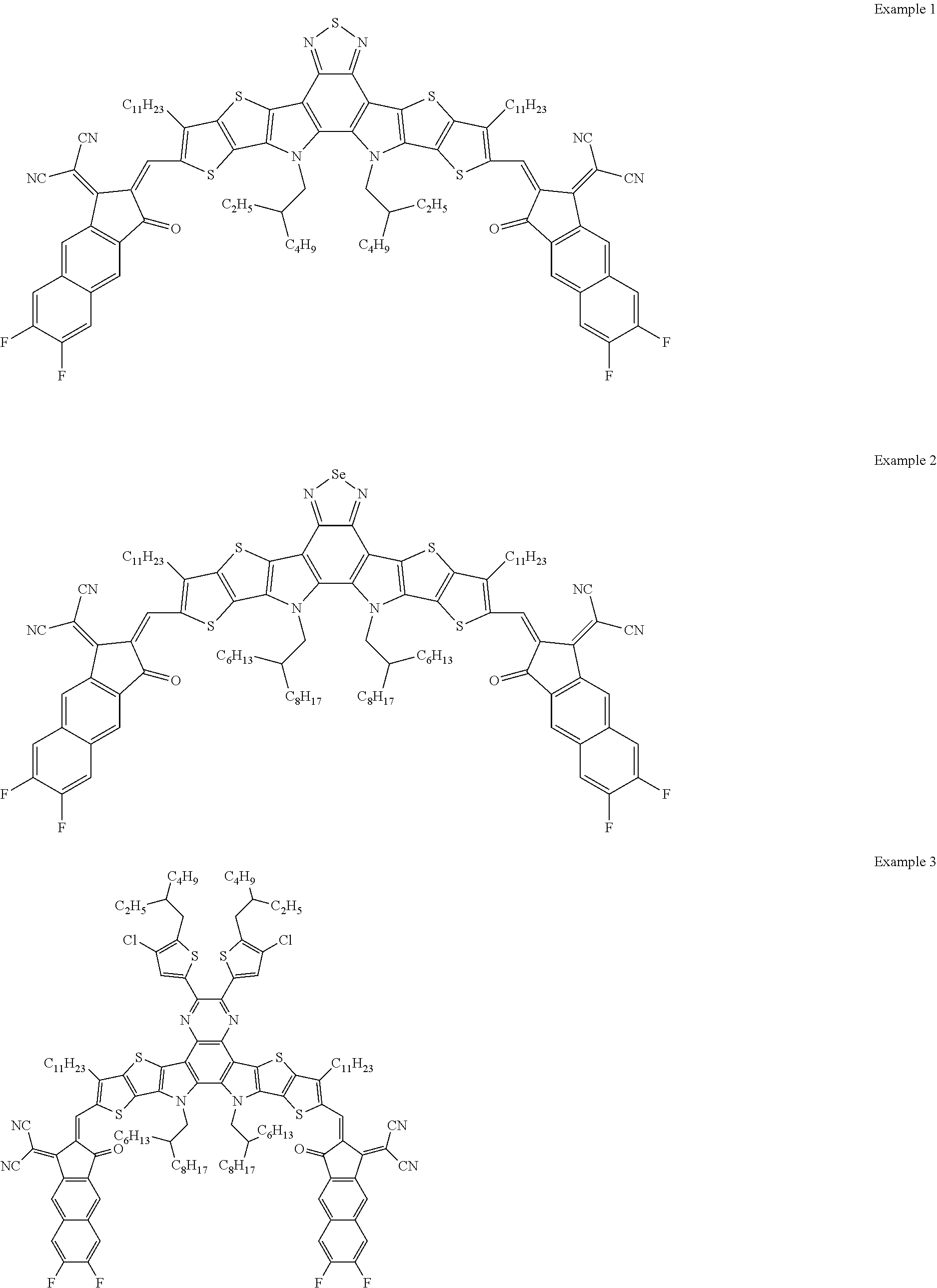

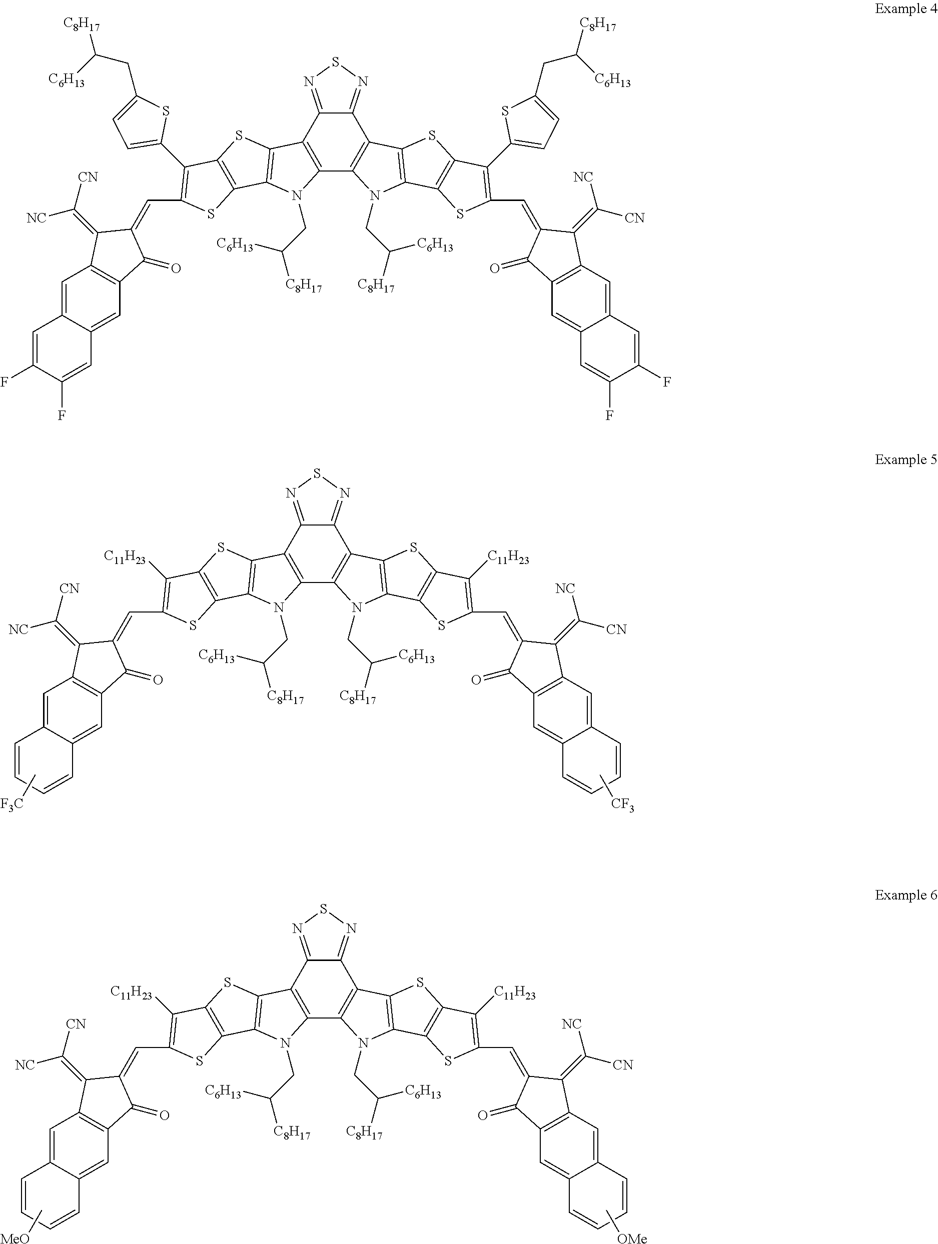

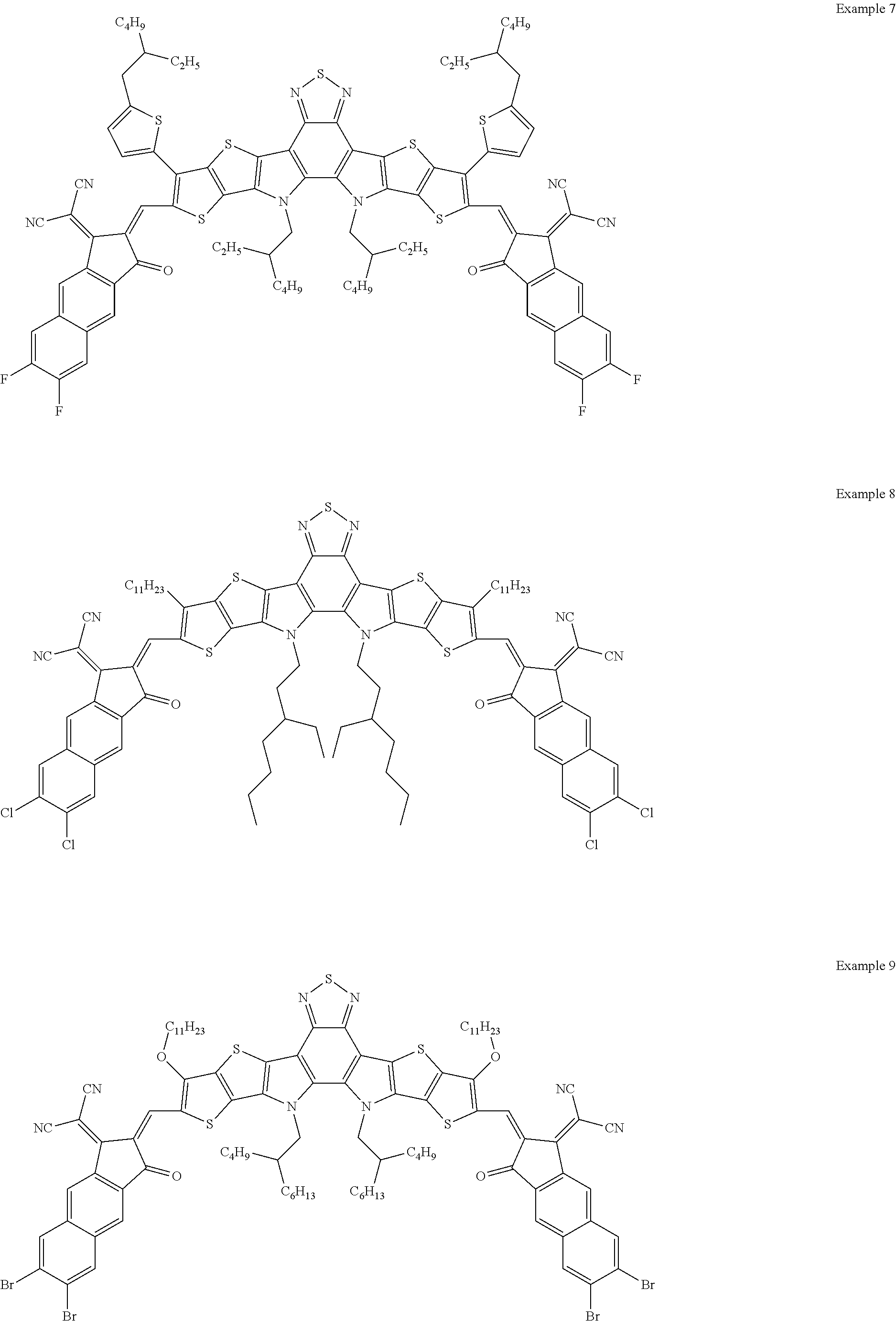

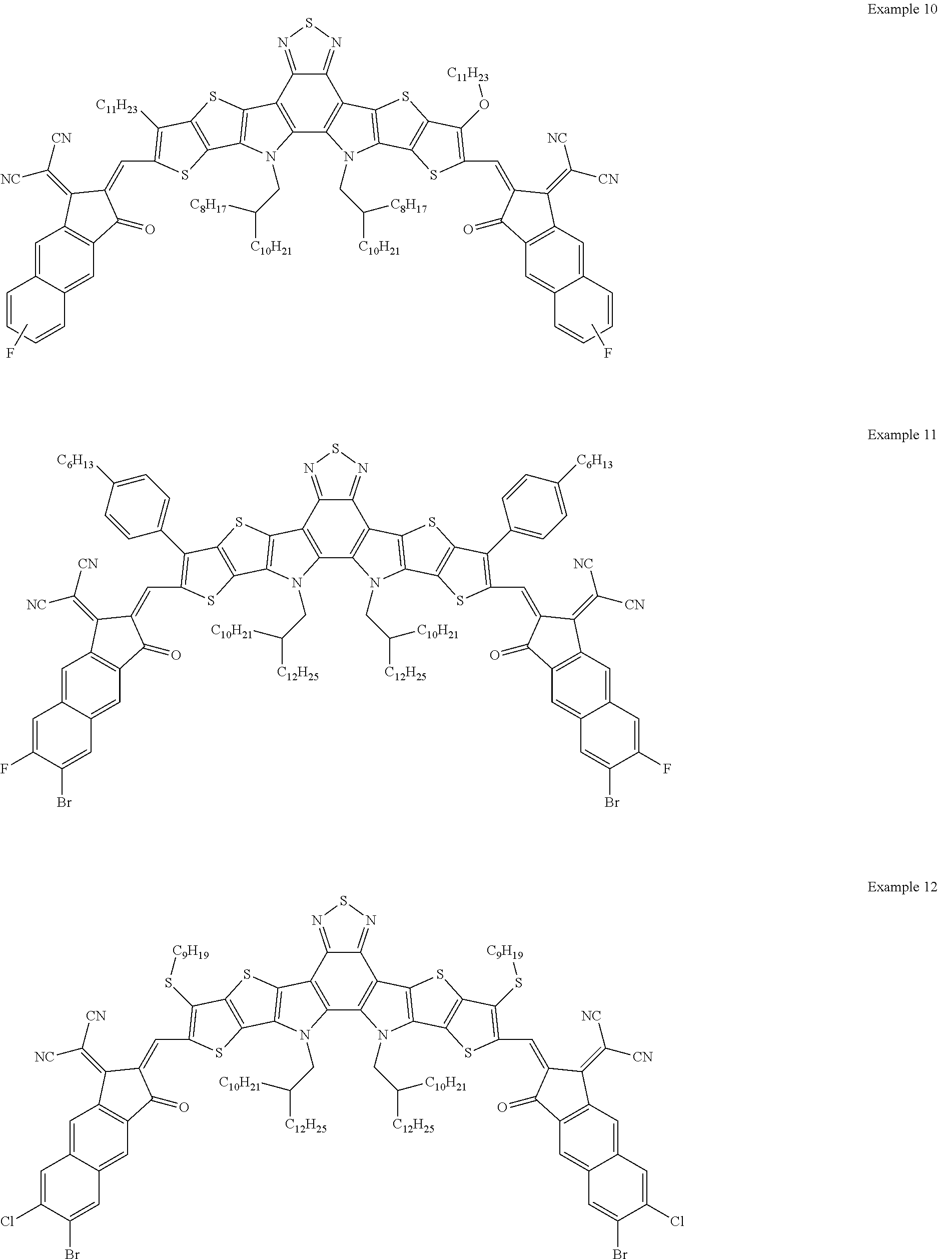

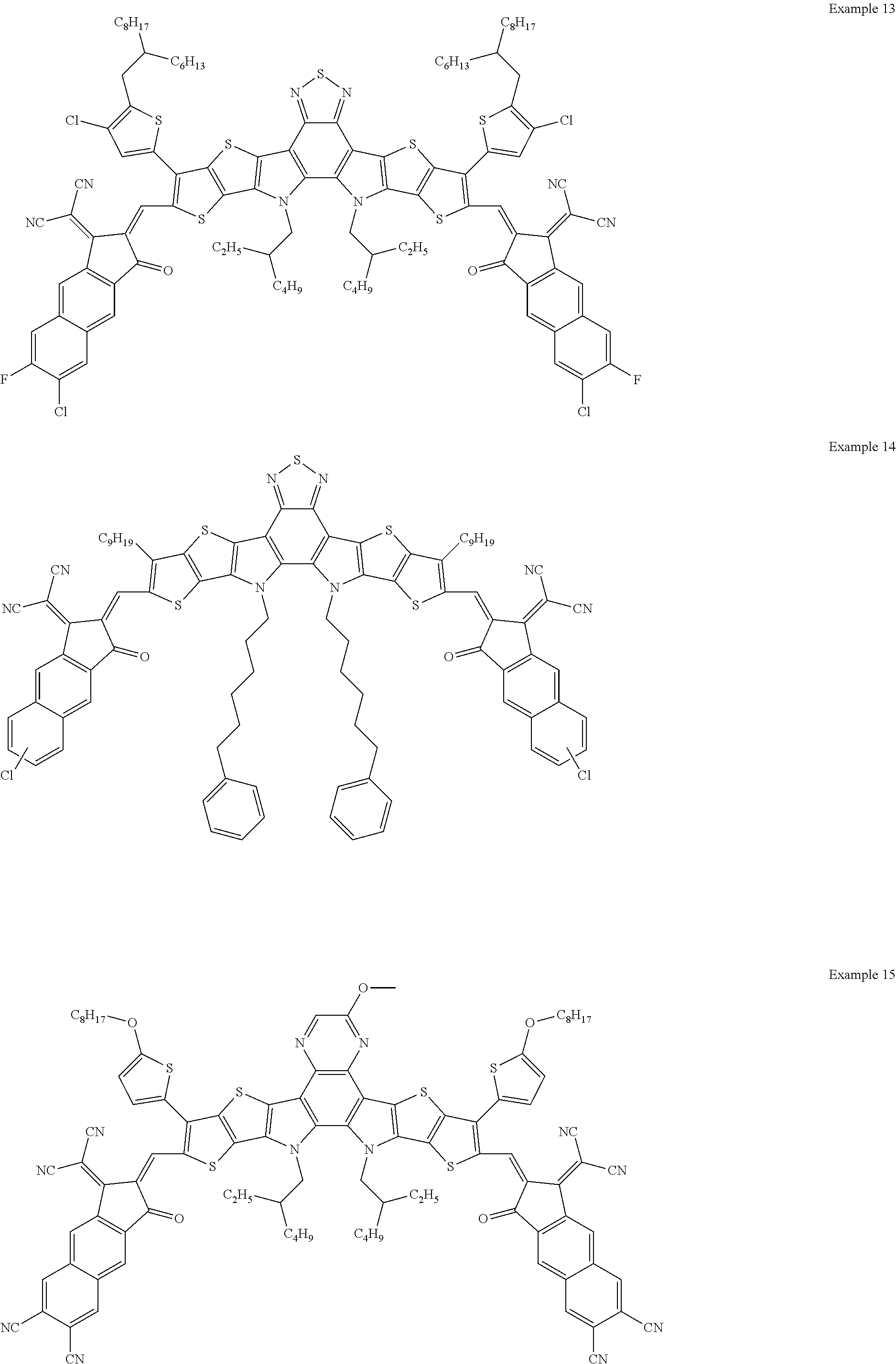

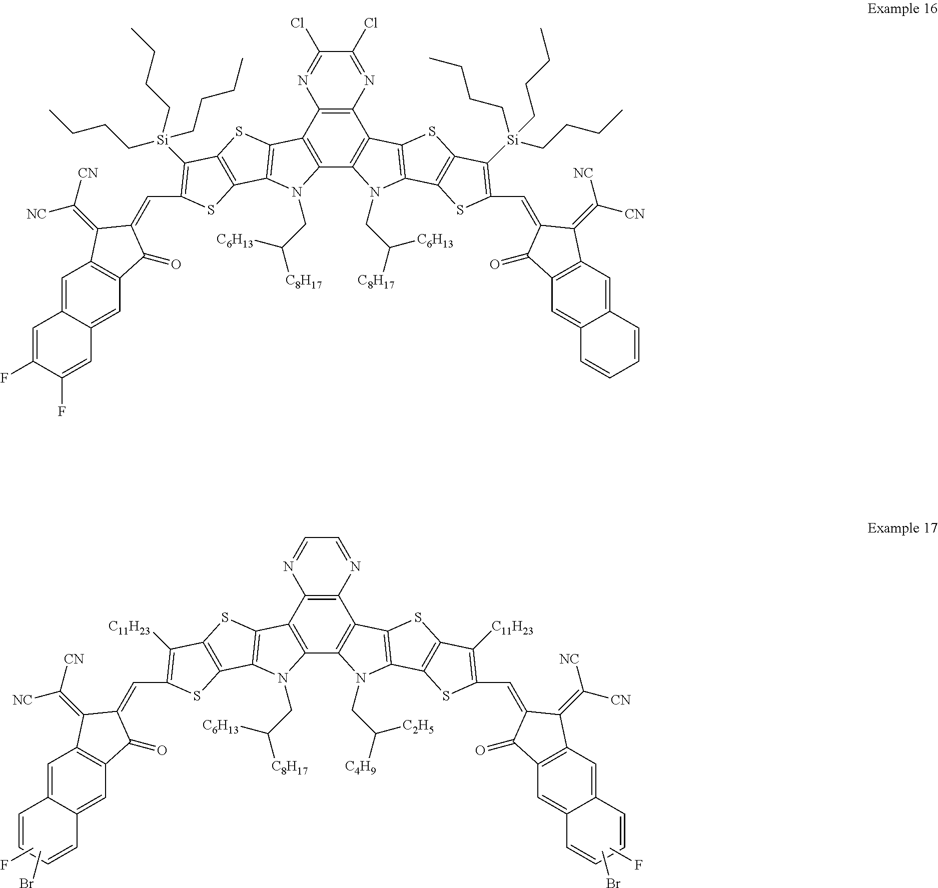

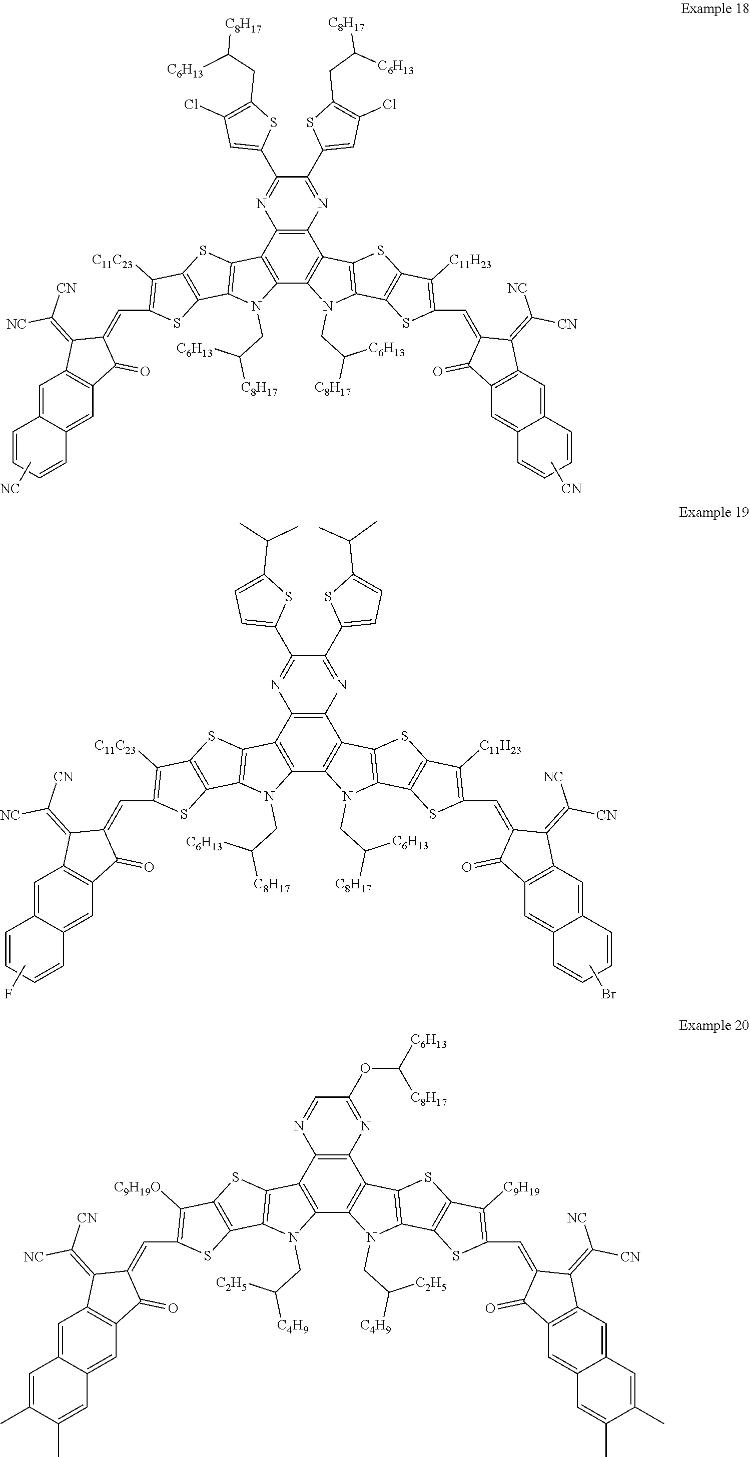

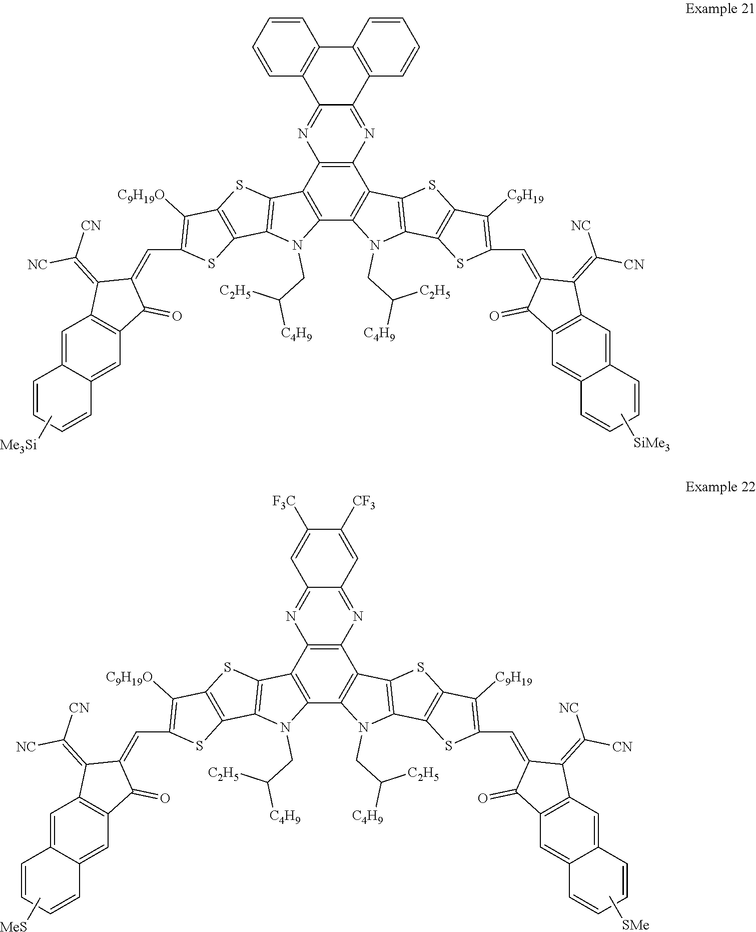

It should be understood that the above-listed embodiments are only intended to allow the person skilled in the art to understand the structure and composition of the present invention more clearly, and are not limited thereto.

In one embodiment, the donor material is further selected from at least one organic conjugated polymer. In practice, the organic conjugated polymer is selected from the following structures:

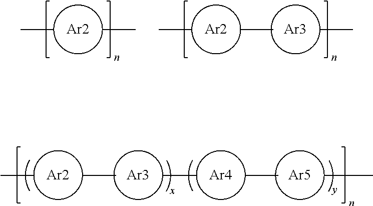

Wherein, Ar2, Ar3, Ar4 and Ar5 are monocyclic or polycyclic structures containing C4-C30 ring atoms respectively. n is the number of molecules, and n is a positive integer from 1 to 1000. x and y are molar fractions, where 0<x<1, 0<y<1 and x+y=1. In a preferred embodiment, at least one of the ring atoms included in Ar2, Ar3, Ar4 and Ar5 is a heteroatom, wherein the heteroatom is independently selected from at least one of S, O, Se, N, F, C1 and Si.

In one embodiment, Ar2 and Ar4 are independently selected from the following structures:

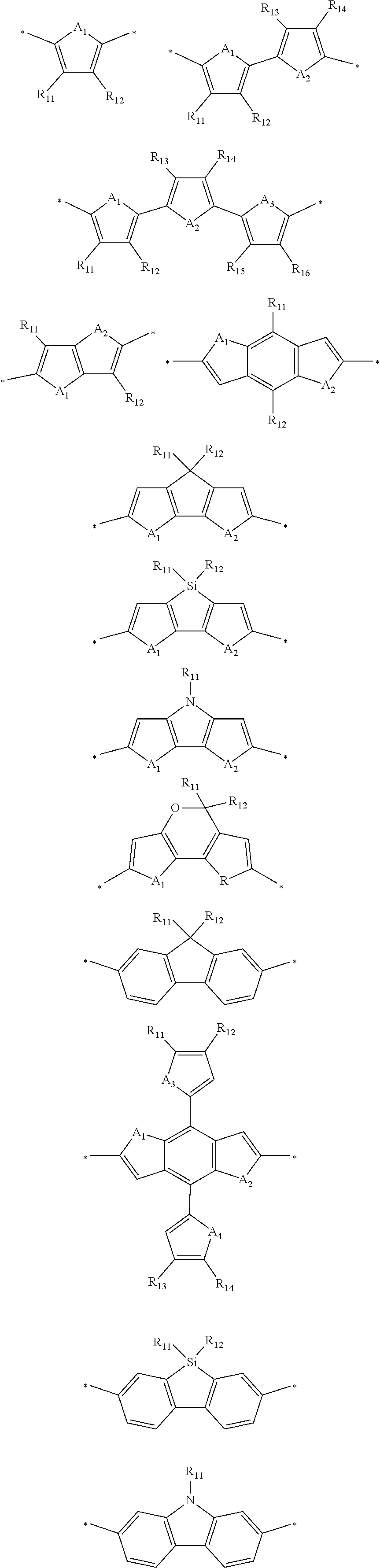

Wherein, A₁, A₂, A₃ and A₄ are independently selected from the following group consisting of O, S, Se and N—R, and R is selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxy heteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl. R₁₁, R₁₂, R₁₃, R₁₄, R₁₅ and R₁₆ are independently selected from the following groups and their derivatives consisting of hydrogen, halogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkylaryl, C1-C30 haloalkyl heteroaryl, C1-C30 esteryl aryl and C2-C30 esteryl heteroaryl.

In one embodiment, Ar3 and Ar5 are independently selected from the following structures:

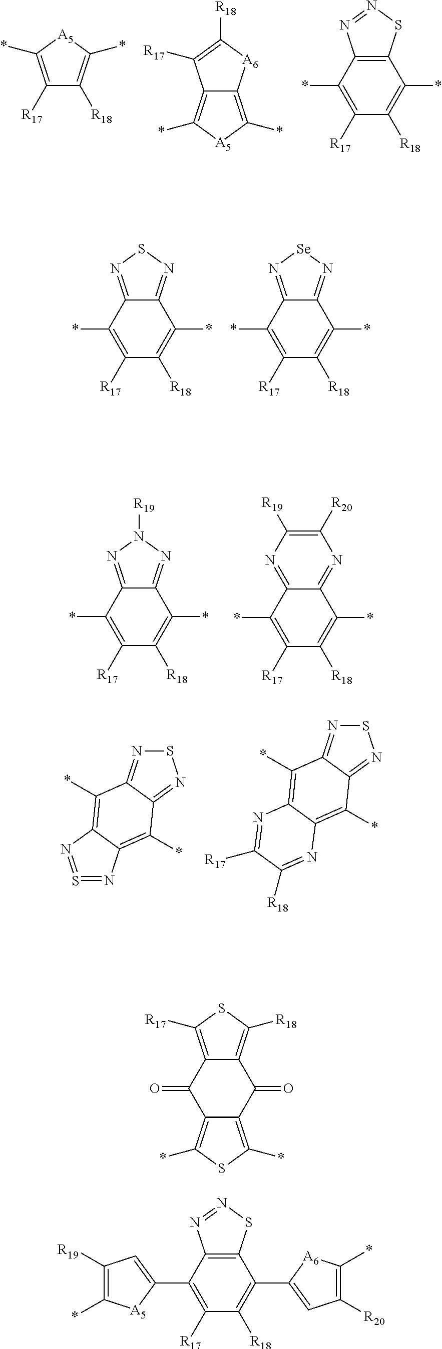

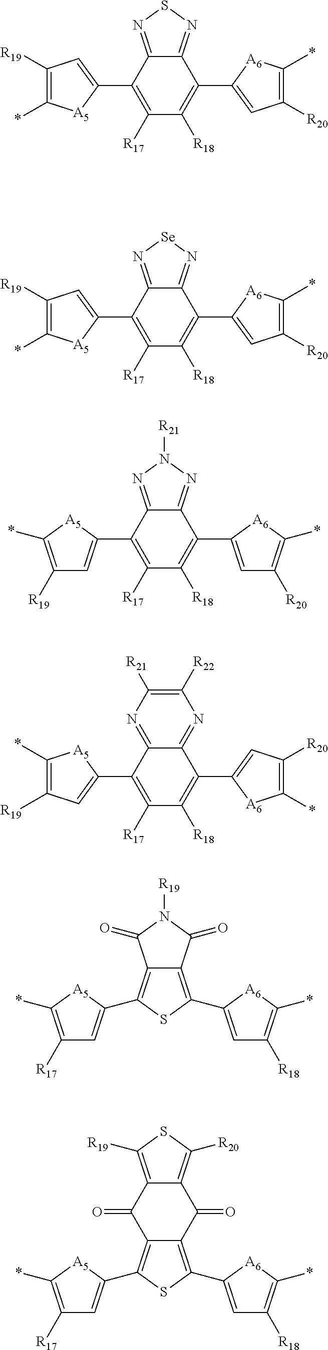

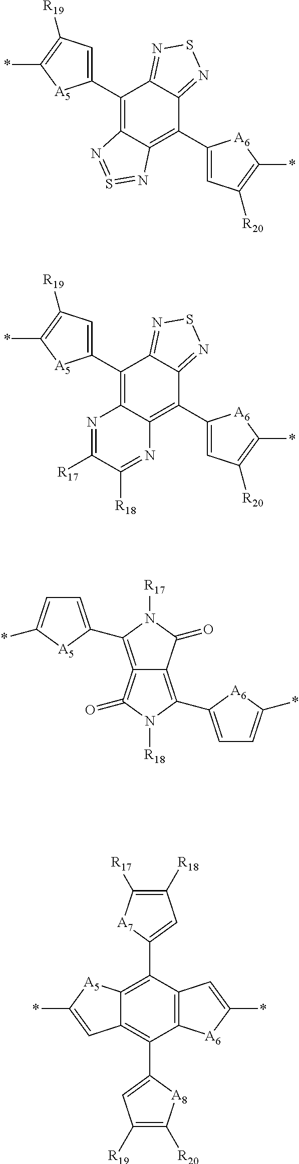

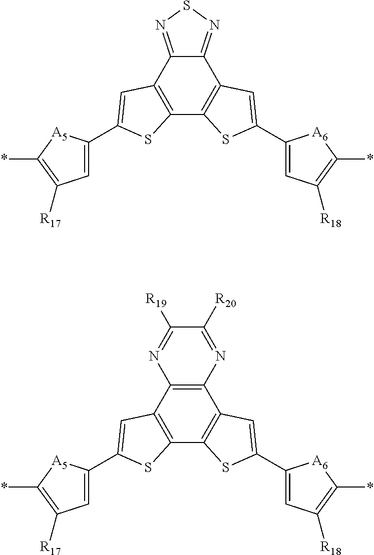

Wherein, A₅, A₆, A₇ and A₈ are independently selected from the following group consisting of O, S, Se and N—R, and R is selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl-, C1-C30 alkyl heteroaryl, C1-C30 silyl aryl, C1-C30 silyl heteroaryl, C1-C30 alkoxy aryl, C1-C30 alkoxy heteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl. R₁₇, R₁₈, R₁₉, R₂₀, R₂₁ and R₂₂ are independently selected from the following groups and their derivatives consisting of hydrogen, halogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silylaryl, C1-C30 silylheteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C1-C30 esteryl aryl and C2-C30 esteryl heteroaryl.

Specifically, when the donor material (p-type material) is an organic conjugated polymer, it can include the following embodiments P-type 1 to P-type 36:

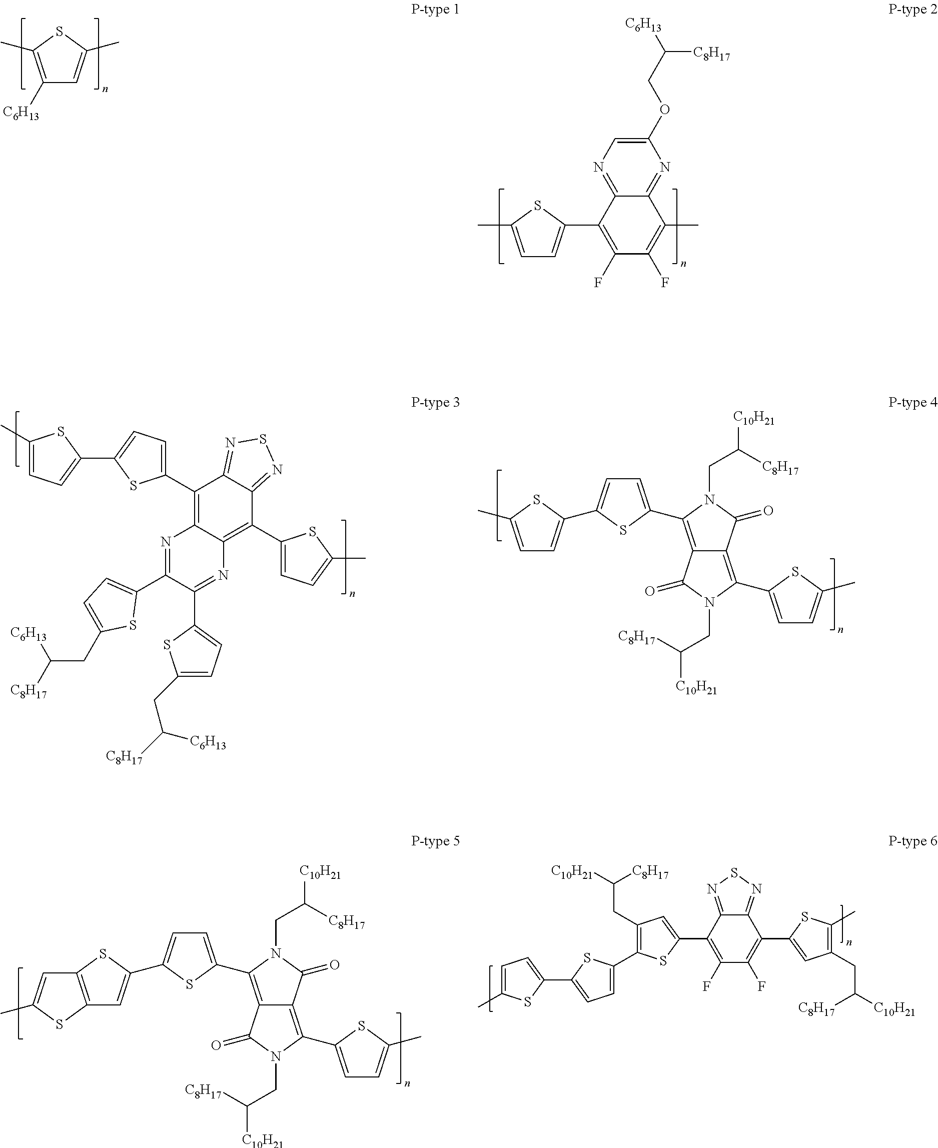

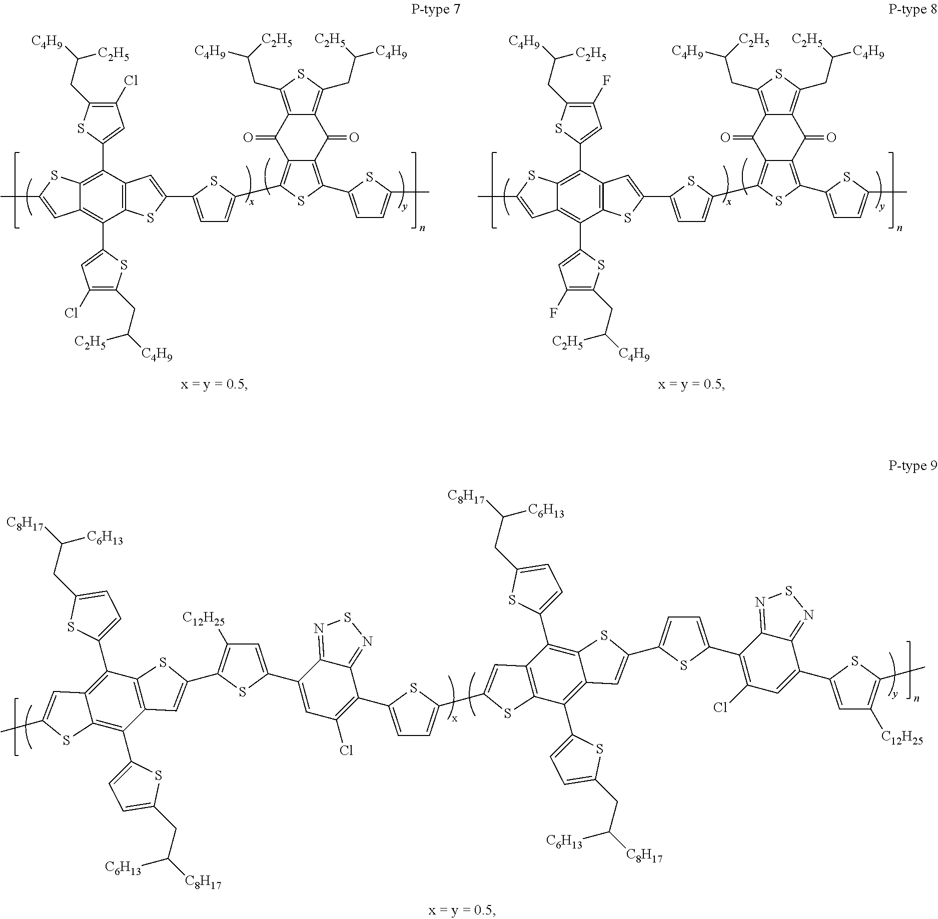

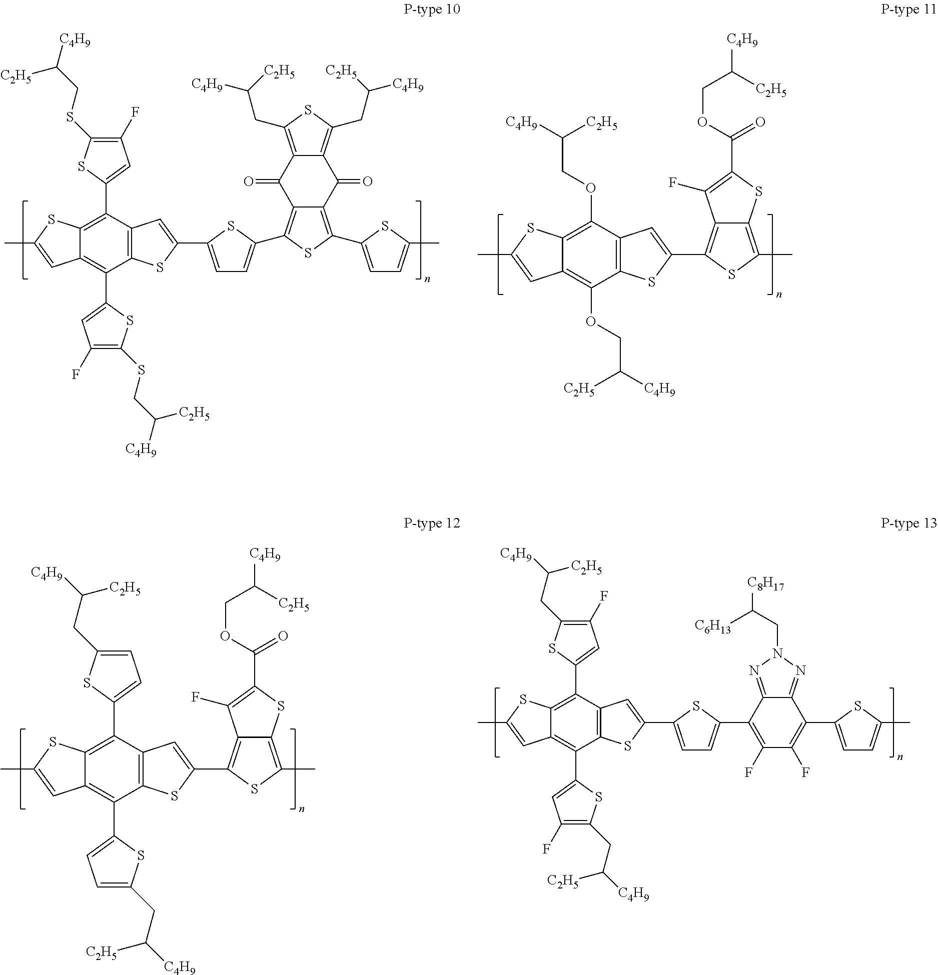

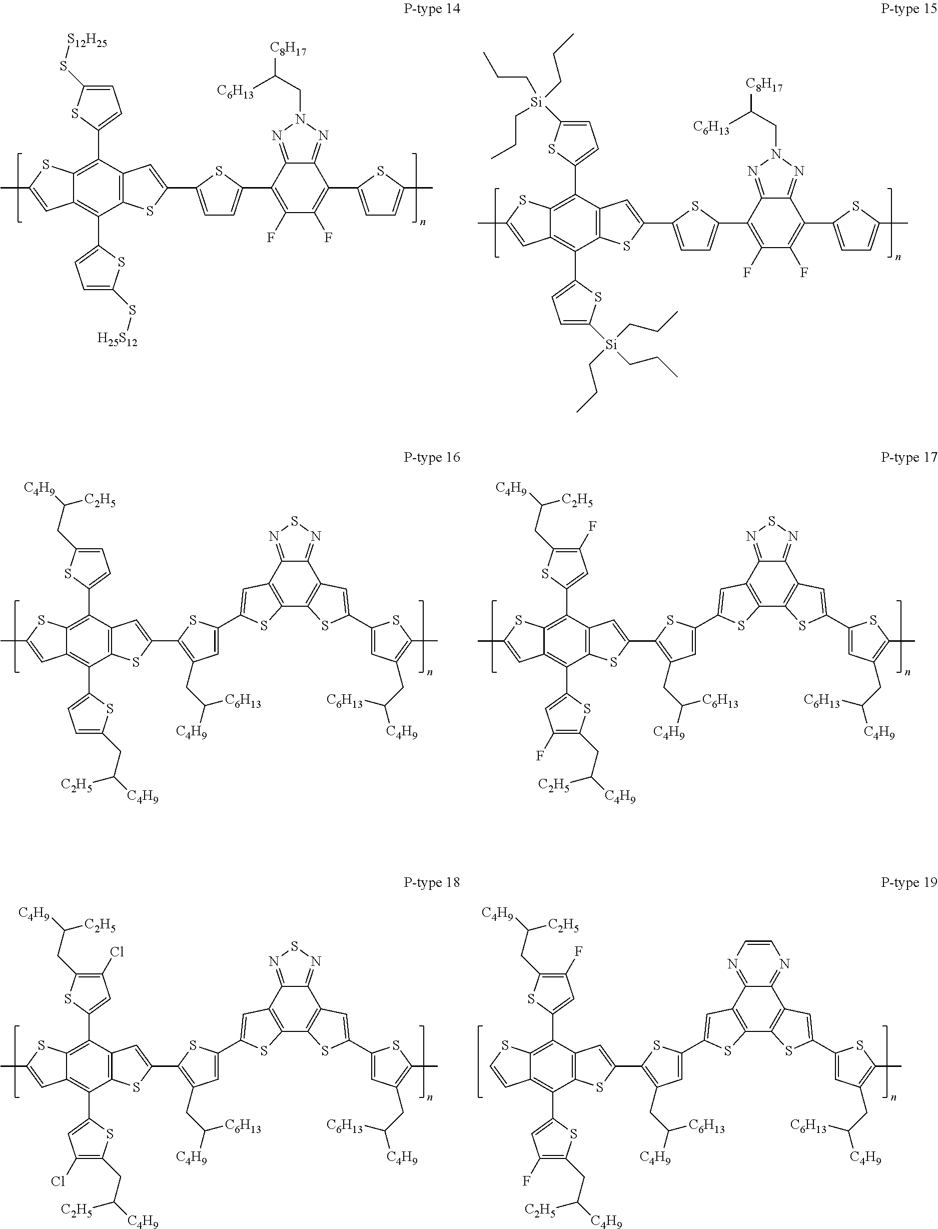

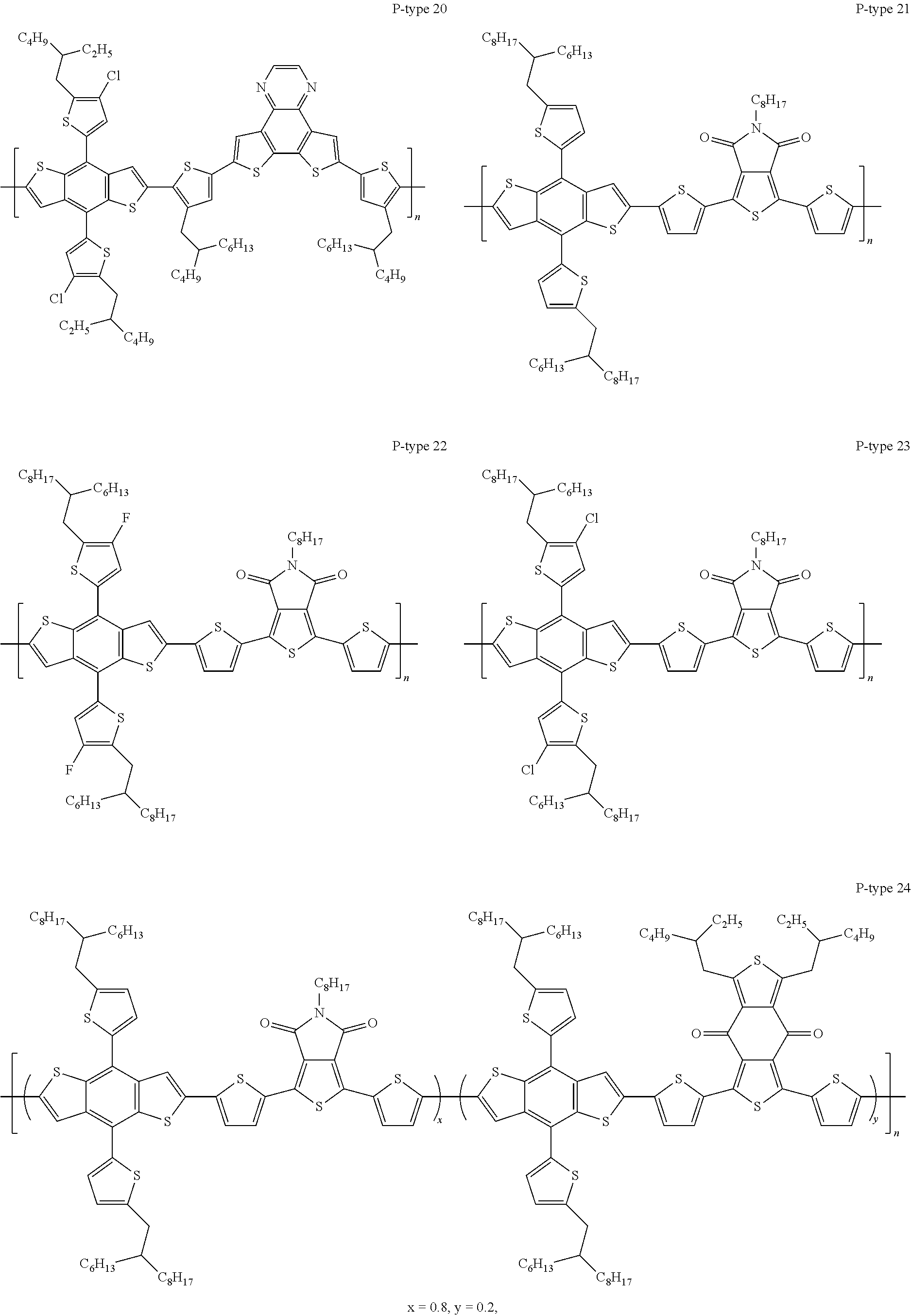

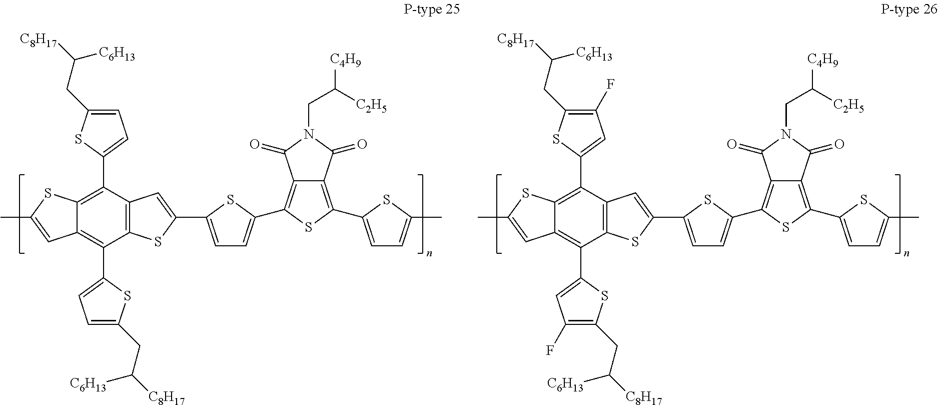

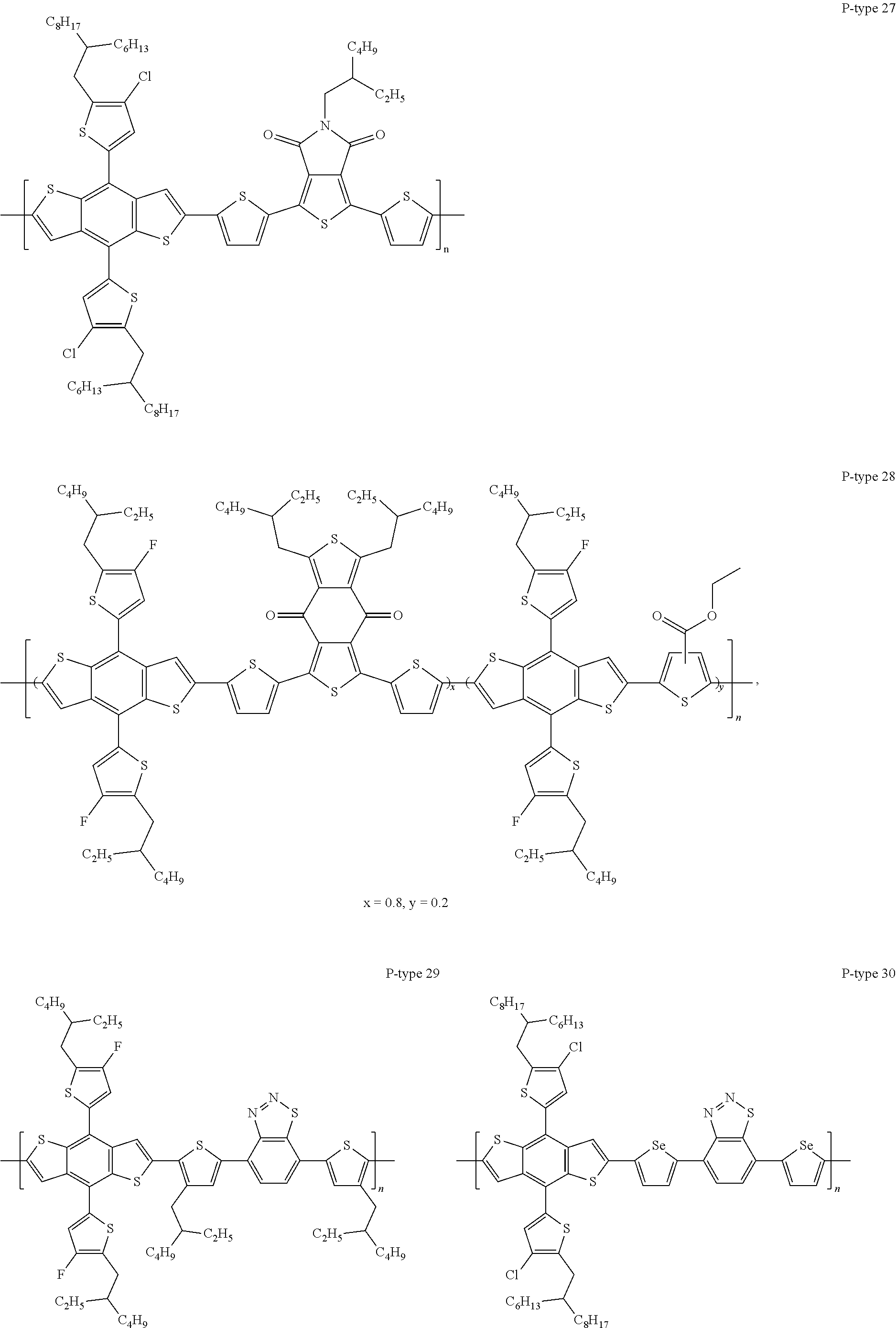

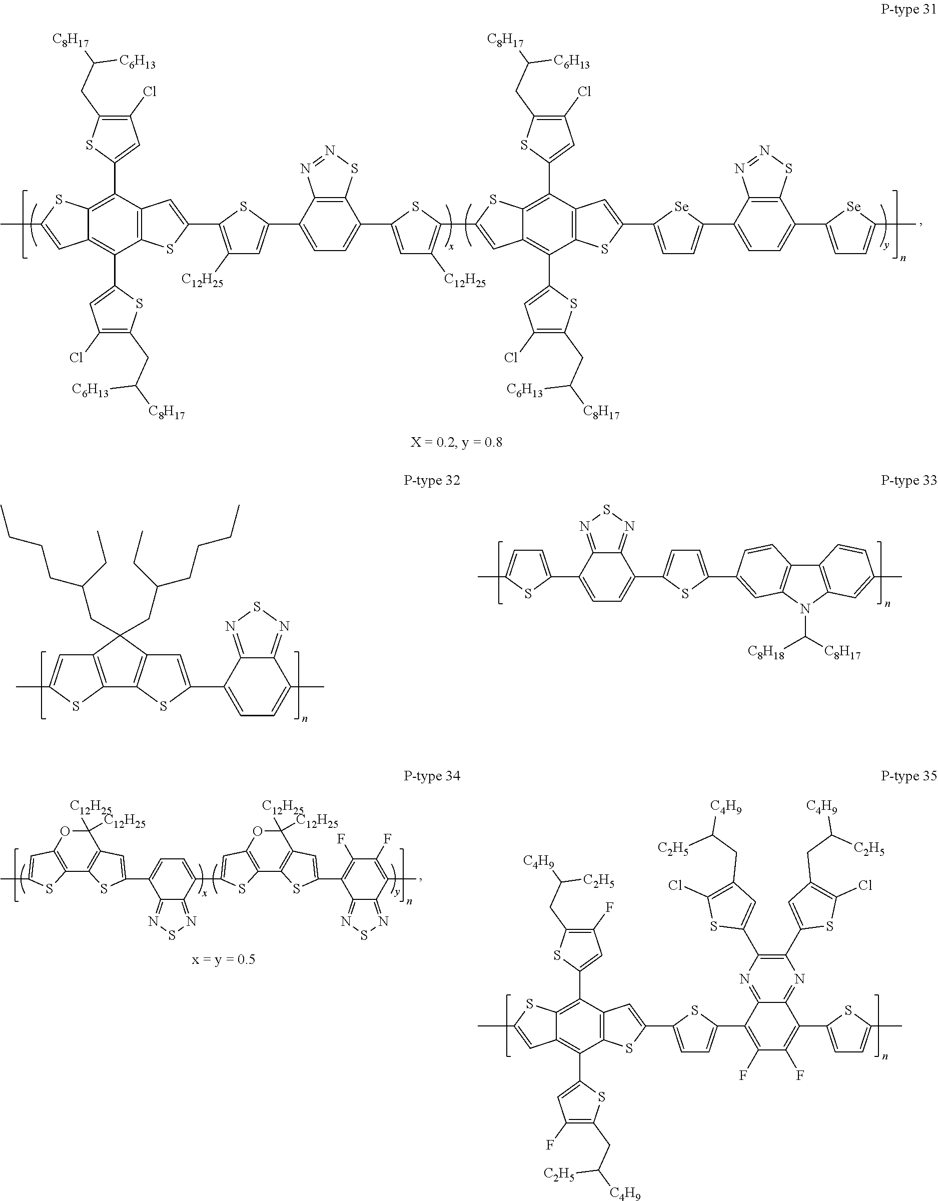

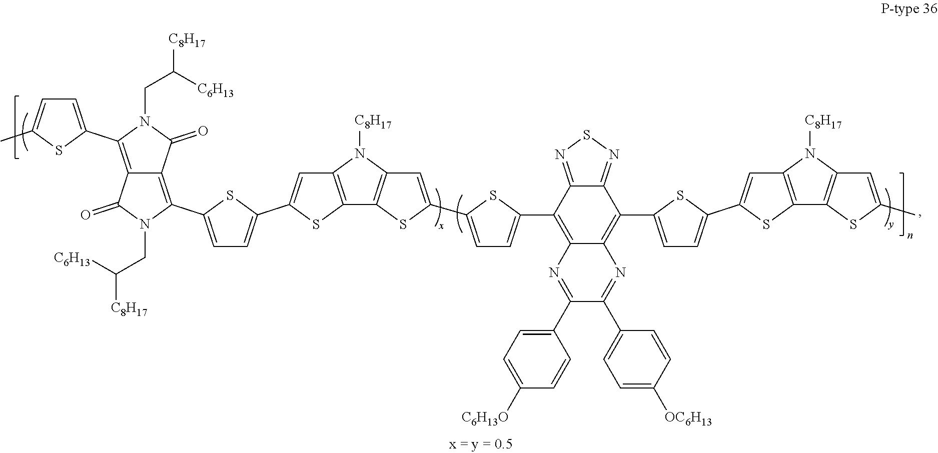

It should be understood that the above-listed embodiments are only intended to allow the person skilled in the art to understand the structure and composition of the present invention more clearly, and are not limited thereto.

In one embodiment, the acceptor material further comprises a second acceptor material, and the second acceptor material is an organic conjugated polymer or an organic conjugated small molecule. In practice, the second acceptor material comprises a fullerene structure. Specifically, the fullerene structure may comprise the following examples of Fullerene 1 to Fullerene 10:

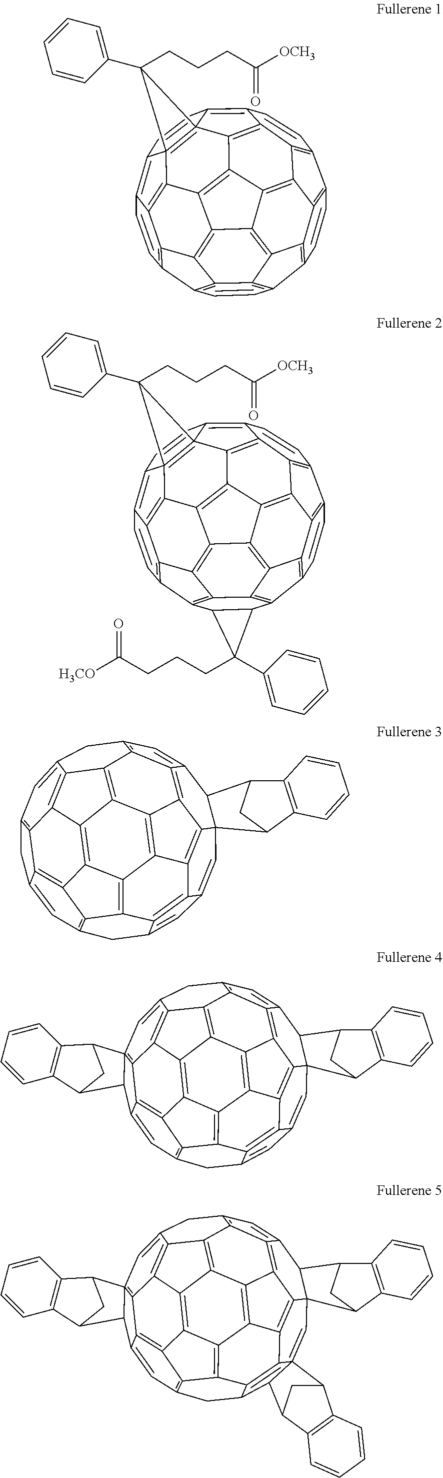

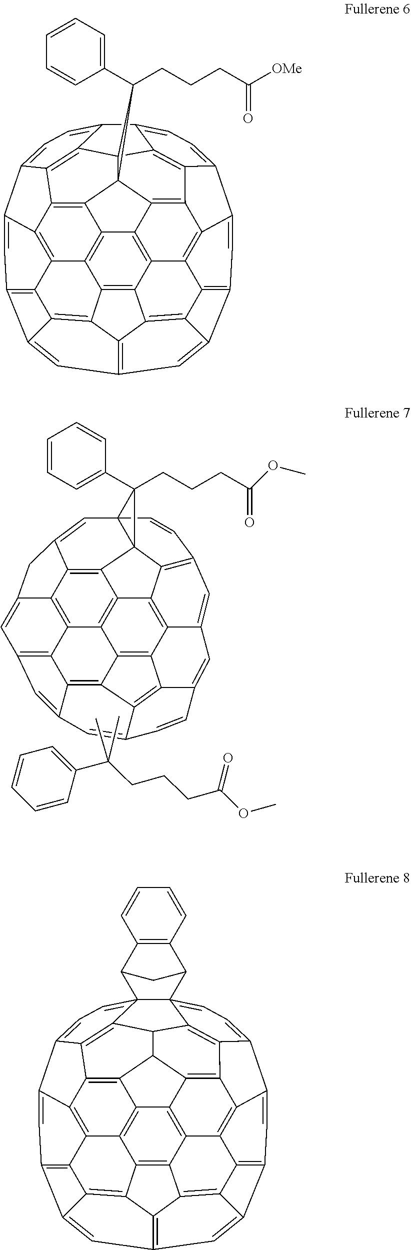

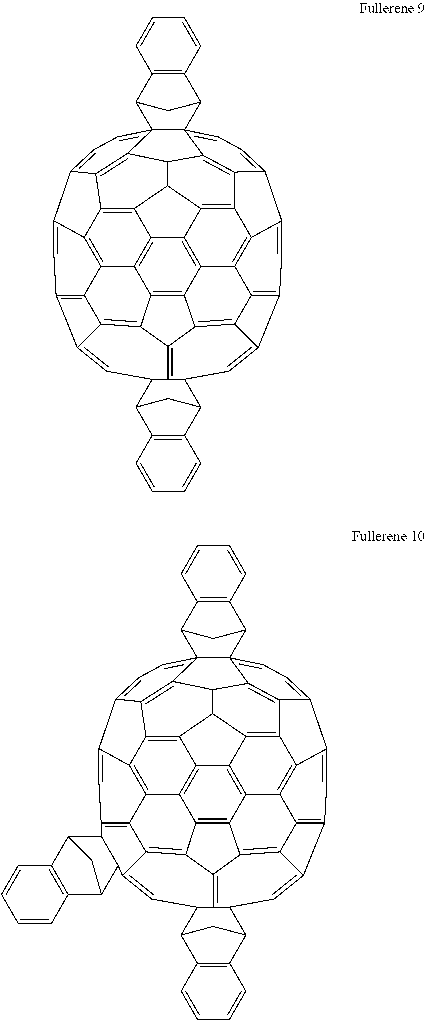

It should be understood that the above-listed embodiments are only intended to allow the person skilled in the art to understand the structure and composition of the present invention more clearly, and are not limited thereto.

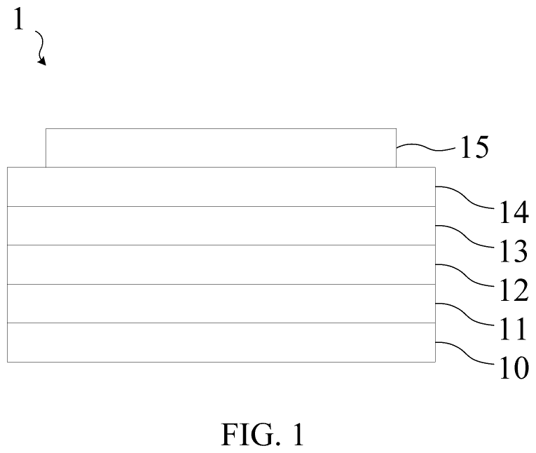

Please refer to FIG. 1. FIG. 1 shows a schematic structural diagram of one embodiment of an organic optoelectronic device of the present invention. As shown in FIG. 1, in another embodiment, the present invention further provides an organic optoelectronic device 1, which comprises a first electrode 11, a second electrode 15 and an active layer 13. The active layer 13, which comprises the aforementioned organic composition comprising Formula I, is disposed between the first electrode 11 and the second electrode 15. The organic optoelectronic device 1 further comprises a first carrier transporting layer 12 and a second carrier transporting layer 14. The organic optoelectronic device 1 may have a stacked structure, which sequentially includes a substrate 10, the first electrode 11 (transparent or semi-transparent electrode), the first carrier transporting layer 12, the active layer 13, the second carrier transporting layer 14 and the second electrode 15. The first carrier transporting layer 12 is configured to transport carriers in the first electrode 11 and the active layer 13, and the second carrier transporting layer 14 is configured to transport carriers in the active layer 13 and the second electrode 15. Specifically, the first carrier transporting layer 12 is one of an electron transporting layer and a hole transporting layer, and the second carrier transporting layer 14 is the other one. In detail, when the first carrier transporting layer 12 is the electron transport layer, the second carrier transporting layer 14 is the hole transport layer, which is an inverted stacked structure; when the first carrier transporting layer 12 is the hole transporting layer, the second carrier transporting layer 14 is an electron transporting layer, which is a conventional stacked structure. In practice, the organic optoelectronic device 1 may comprise an organic photovoltaic device, an organic photodetector device, or an organic light emitting diode.

In order to illustrate the organic composition of the present invention more clearly, the following experiments will be performed to illustrate the difference in efficacy between Comparative example 1 and the aforementioned Example 1 to Example 5 of the present invention. Then, they are further used as first acceptor materials to prepare active layers and organic optoelectronic devices for material testing and device testing.

For the optical physical quality testing part of material testing and device testing, the UV absorption spectrum measurement instrument model is Hitachi UH5700, and the oxidation potential is measured by using cyclic voltammetry with CH Instrument 611E.

### Preparation of the First Acceptor Material:

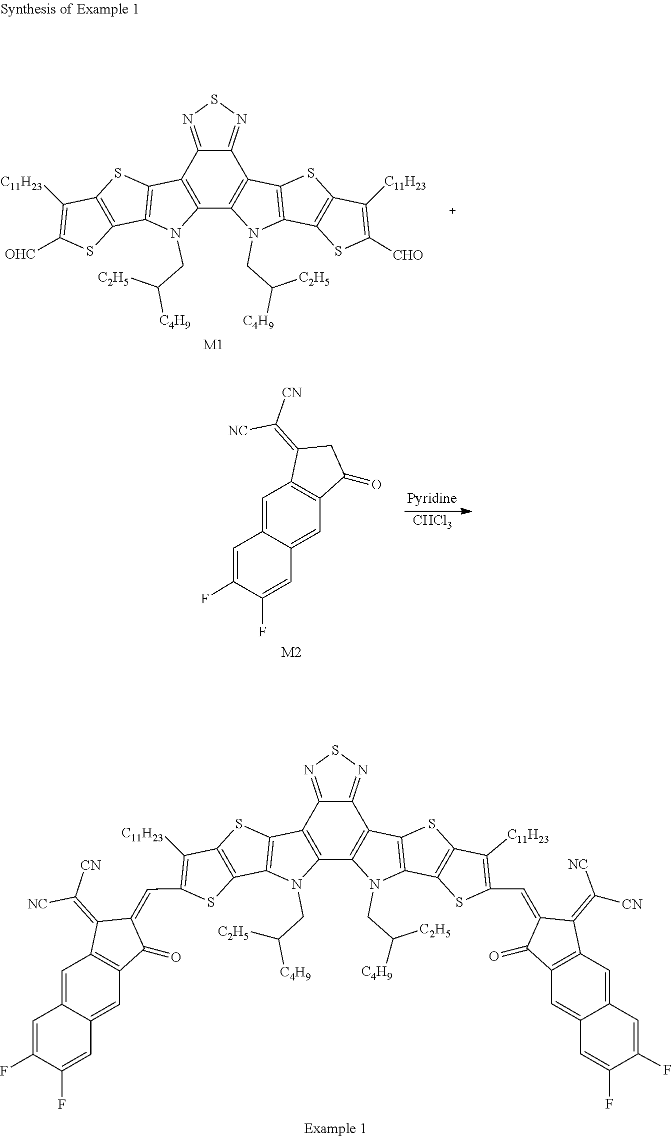

M1 (150 mg, 0.141 mmol), M2 (118 mg, 0.422 mmol) and chloroform (CF, 7.2 mL) were placed into a 100 mL three-necked reaction flask and was stirred with a magnet. Pyridine (0.08 mL) was added and the temperature of the reaction mixture was heated to 60° C. and reacted for 6 hours. Methanol was added to precipitate the product, and the solid was collected by suction filtration. The crude product was purified by a silica gel column, and the eluent was heptane/chloroform=1/1, and the product Example 1 (140 mg, yield: 62%) was obtained as a blue-black solid. ¹H NMR (600 MHz, CDCl₃): δ 9.27 (s, 2H), 9.14 (s, 2H), 8.34 (s, 2H), 7.87-7.80 (m, 4H), 4.80 (d, J=4.2 Hz, 4H), 3.27 (t, J=9.0 Hz, 4H), 2.12 (br, 2H), 1.92-1.88 (m, 4H), 1.52-0.87 (m, 48H), 0.78-0.77 (m, 6H), 0.77-0.69 (m, 6H), 0.68-0.67 (m, 6H).

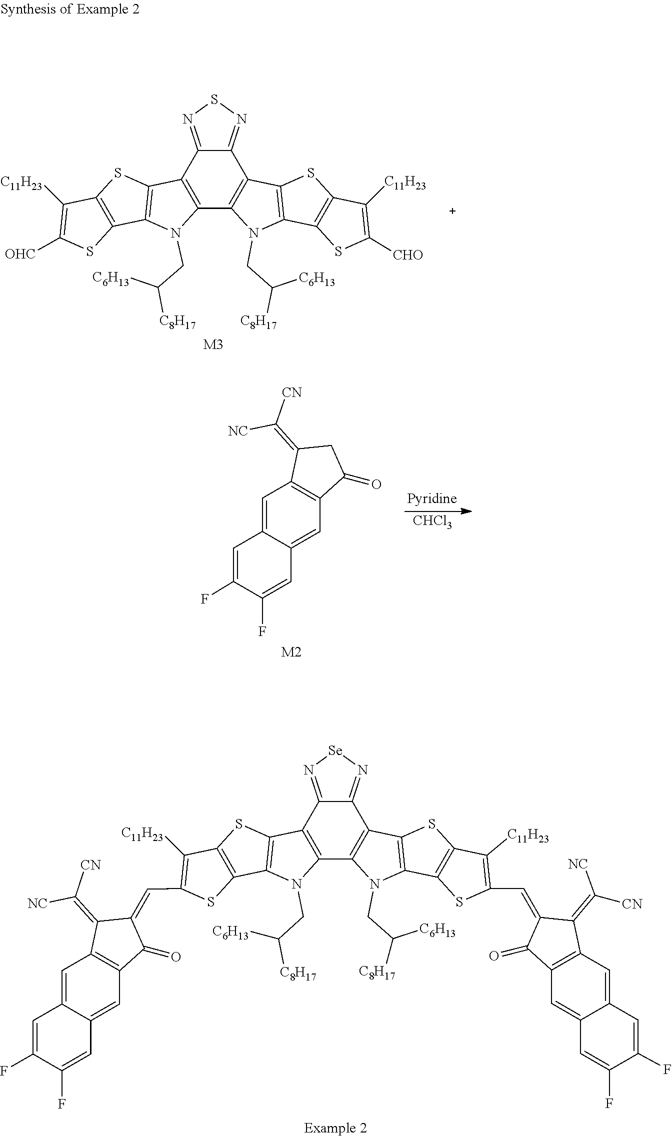

M3 (357 mg, 0.275 mmol), M2 (308 mg, 1.099 mmol) and chloroform (CF, 10 mL) were placed into a 100 mL three-necked reaction flask and was stirred with a magnet. Pyridine (0.2 mL) was added and the temperature of the reaction mixture was heated to 60° C. and reacted for 4 hours. Methanol was added to precipitate the product, and the solid was collected by suction filtration. The crude product was purified by a silica gel column, and the eluent was heptane/chloroform=1/1, and the product Example 2 (410 mg, yield: 82%) was obtained as a blue-black solid. ¹H NMR (500 MHz, CDCl₃): δ 9.26 (s, 2H), 9.14 (s, 2H), 8.32 (m, 2H), 7.87-7.78 (m, 4H), 4.74 (d, J=12 Hz, 4H), 3.26 (t, J=7.5 Hz, 4H), 2.16 (br, 2H), 1.90 (t, J=7.5 Hz, 4H), 1.57-0.85 (m, 86H), 0.79-0.76 (m, 6H), 0.71-0.69 (m, 6H).

### Synthesis of Example 3

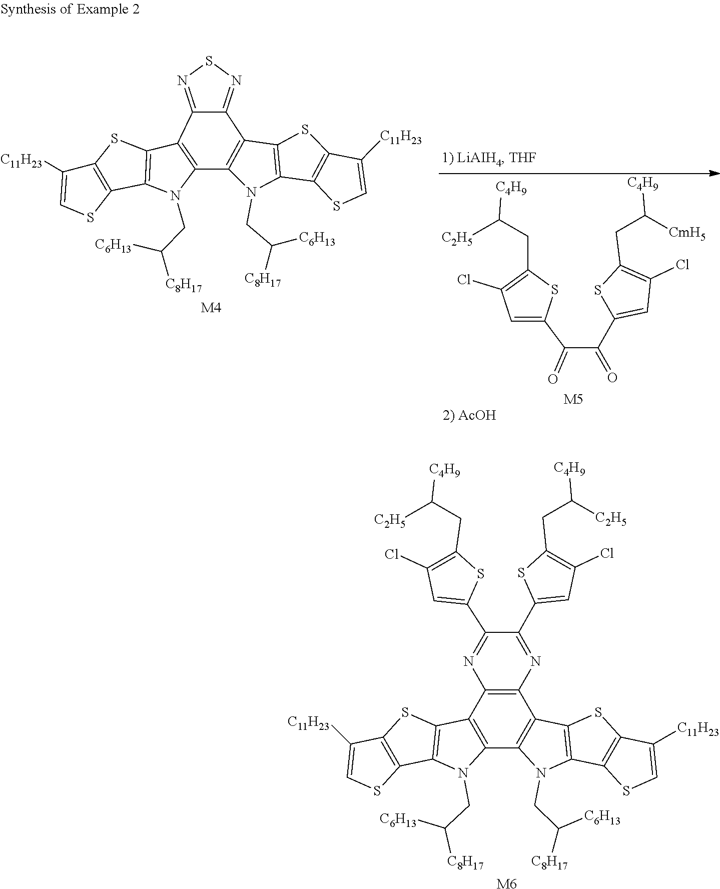

M4 (2.0 g, 1.67 mmol) was placed into a 100 mL three-necked reaction flask and anhydrous tetrahydrofuran (THF, 40 mL) was added in. Lithium aluminum hydride (LiAlH₄, 0.64 g, 16.7 mmol) was added under ice bath. After the reaction mixture stopped bubbling, the temperature of the reaction was warmed to room temperature. After the reaction mixture was reacted for 2 hours, and the reaction mixture was quenched by slowly dropping deionized water. The mixture was extracted with heptane/H₂O. The organic layer was collected and dried with MgSO₄, and the organic solvent was removed to obtain an intermediate product. The intermediate product was placed into a 100 mL three-necked reaction flask and was dissolved with acetic acid (55 mL). M5 (0.78 g, 1.52 mmol) was placed in, and the temperature of the reaction mixture was heated to 120° C. for 2 hours. After the reaction of the reaction mixture was completed, the temperature of the reaction was cooled to room temperature. The mixture was extracted three times with dichloromethane/water (DCM/H₂O). The organic layer was dried with MgSO₄, and the organic solvent was removed to obtain crude product. The crude product was purified by a silica gel column, and the eluent was heptane, and the product M6 (2.0 g, yield: 86%) was obtained as a red oil. ¹H NMR (600 MHz, CDCl₃): δ 7.33 (s, 2H), 7.02 (s, 2H), 4.52 (d, J=7.8 Hz, 4H), 2.85-2.90 (m, 8H), 1.81-1.92 (m, 6H), 0.76-1.57 (m, 122H), 0.70 (t, J=6.6 Hz, 6H).

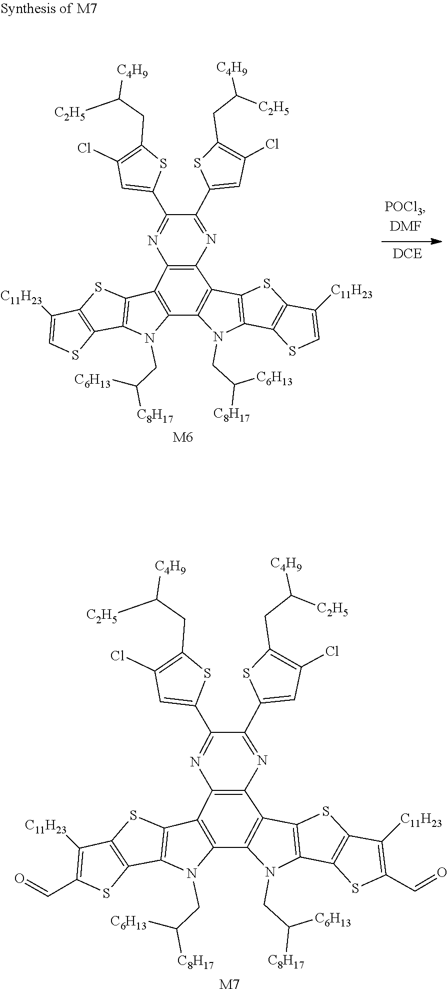

Phosphorus oxychloride (POCl₃, 0.26 mL, 2.75 mmol) and dimethylformamide (DMF, 1.78 mL, 22.9 mmol) were mixed in a 100 ml two-necked reaction flask in an ice bath, and were stirred with a magnet for 30 minutes to form Vilsmeier reagent. Prepare another 100 ml two-necked reaction flask, M6 (756 mg, 0.46 mmol) and dichloroethane (DCE, 30 mL) were placed in and were stirred with a magnet. Then, the Vilsmeier reagent was added in, and the temperature of the reaction mixture was heated to 70° C. for 18 hours. The temperature of the reaction was cooled to room temperature, and the mixture was extracted three times with dichloromethane/water (DCM/H₂O). The organic layer was collected and was dried with MgSO₄, and the organic solvent was removed. The crude product was purified by a silica gel column, and the eluent was heptane/chloroform=1/1, and the product M7 (437 mg, yield: 56%) was obtained as an orange-red solid. ¹H NMR (600 MHz, CDCl₃): δ 10.15 (s, 2H), 7.34 (s, 2H), 4.66 (d, J=7.8 Hz, 4H), 3.23 (t, J=7.8 Hz, 4H), 2.85-2.92 (m, 4H), 2.09 (m, 2H), 1.95 (m, 4H) 1.80 (d, J=12.6 Hz, 2H), 0.76-1.43 (m, 120H), 0.69 (t, J=7.2 Hz, 6H).

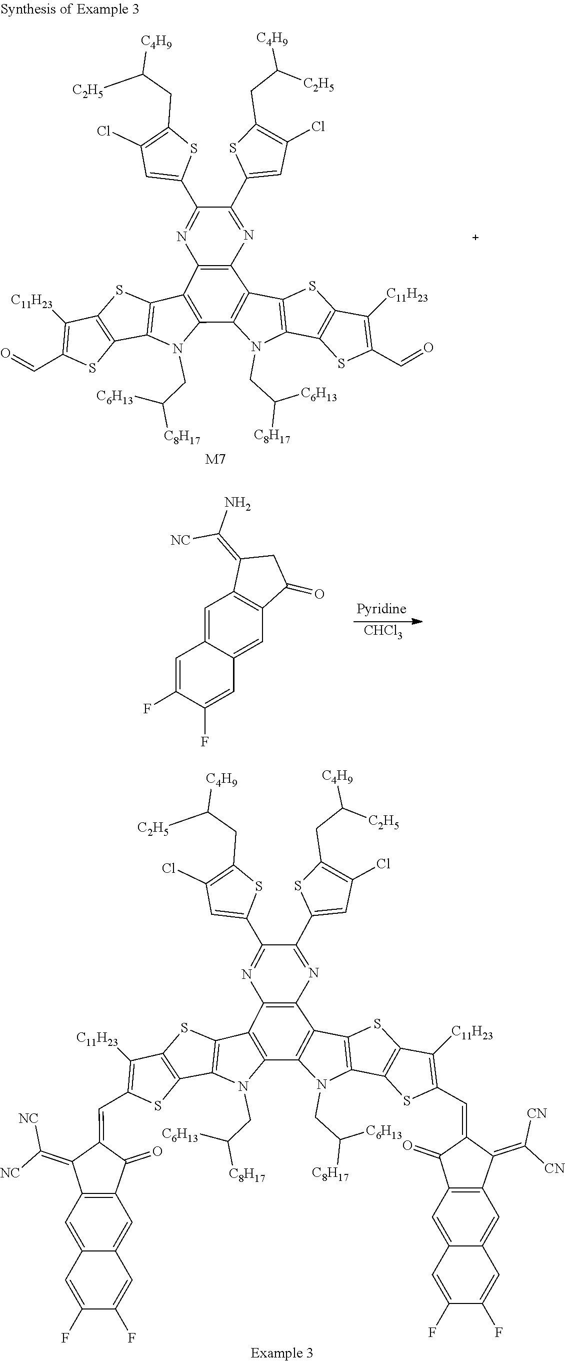

M7 (200 mg, 0.12 mmol), M2 (98.7 mg, 0.36 mmol) and chloroform (CF, 6 mL) were placed into a 100 mL three-necked reaction flask and was stirred with a magnet. Pyridine (0.1 mL) was added and the temperature of the reaction mixture was heated to 60° C. and reacted for 4 hours. Methanol was added to precipitate the product, and the solid was collected by suction filtration. The crude product was purified by a silica gel column, and the eluent was heptane/chloroform=1/2, and the product Example 3 (190 mg, yield: 73%) was obtained as a blue-black solid. ¹H NMR (600 MHz, CDCl₃): δ 9.29 (s, 2H), 9.15 (s, 2H), 8.32 (s, 2H), 7.79-7.88 (m, 4H), 7.43 (s, 2H), 7.04 (d, J=3.6 Hz, 2H), 4.81 (d, J=7.8 Hz, 4H), 3.31 (t, J=7.8 Hz, 4H), 2.86-2.91 (m, 4H), 2.18 (m, 2H), 1.92 (m, 4H), 1.72 (m, 2H), 1.42-0.74 (m, 112H), 0.75 (t, J=7.2 Hz, 6H), 0.71 (t, J=6.9 Hz, 6H).

### Synthesis of Example 4

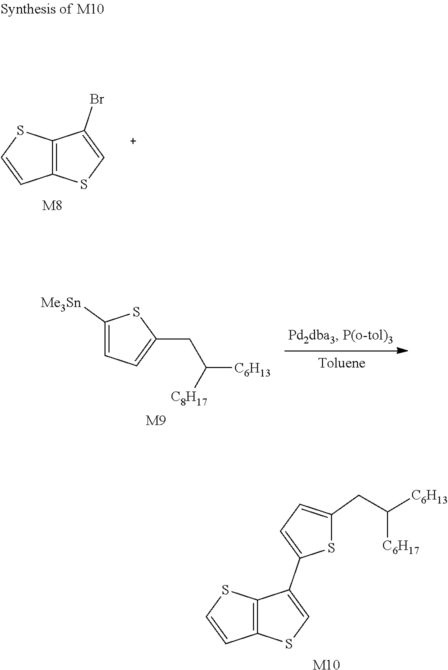

M8 (2.0 g, 9.1 mmol) and M9 (4.3 g, 9.1 mmol) were placed into a 100 mL three-necked reaction flask. Toluene (65 mL) was added in and the reaction mixture was stirred with a magnet. The reaction mixture was deoxygenated at room temperature for 30 minutes. Tris(dibenzylideneacetone)dipalladium (Pd₂dba₃, 333 mg, 0.36 mmol) and tris(o-methylphenyl)phosphine (P(o-tolyl)₃, 443 mg, 1.45 mmol) were added in and the reaction mixture was heated to 80° C. and was reacted for 18 hours. The temperature of the reaction was cooled to room temperature. The crude product was purified by a silica gel column, and the eluent was heptane, and the oil product M10 (3.6 g, 88%) was obtained. ¹H NMR (600 MHz, CDCl₃): δ 7.45 (dd, J=5.1 Hz, J=1.5 Hz, 1H), 7.40 (d, J=1.8 Hz, 1H), 7.29 (d, J=5.4 Hz, 1H), 7.22 (d, J=3.6 Hz, 1H), 6.74 (d, J=3.6 Hz, 1H), 2.77 (d, J=6.0 Hz, 2H), 1.66 (m, 1H), 1.32-1.27 (m, 24H), 0.90-0.86 (m, 6H).

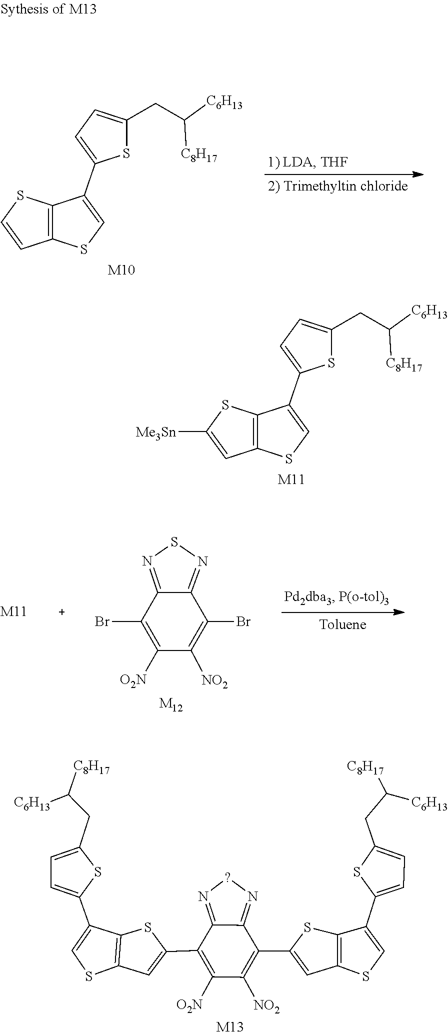

M10 (3.07 g, 6.87 mmol) was placed into a 250 mL three-necked reaction flask, and anhydrous tetrahydrofuran (THF, 60 mL) was added in and was placed it in a −60° C. ice bath with magnet stirring. Lithium diisopropylamide (LDA, 2.0 M, 3.44 mL, 6.87 mmol) was added slowly under ice bath, and then the reaction mixture was reacted at −60° C. for 30 minutes. Then, Trimethyltin chloride (1.37 g, 6.87 mmol, completely dissolved in 5 mL THF) was added slowly at −60° C., and the reaction mixture was warmed to room temperature for 30 minutes. Methanol (MeOH) was slowly added to quench the reaction until bubbling ceased. The mixture was extracted three times with heptane/H₂O. The organic layer was collected and was dried with magnesium sulfate (MgSO₄) to obtain the crude product M11. M11 was carried directly to the next step without additional purification. The crude product M11 and M12 (0.82 g, 2.14 mmol) were placed into a 250 mL three-necked reaction flask, and toluene was added in and stirred with a magnet. The reaction mixture was deoxygenated at room temperature for 30 minutes. Tris(dibenzylideneacetone)dipalladium (Pd₂dba₃, 34 mg, 0.038 mmol) and tris(o-methylphenyl)phosphine (P(o-tolyl)₃, 46 mg, 0.152 mmol) were added in and the reaction mixture was heated to 80° C. and was reacted for 4 hours. The temperature of the reaction was cooled to room temperature. Methanol was added to precipitate the product, and the product red solid M13 (1.94 g, yield: 81%) was collected by suction filtration. ¹H NMR (600 MHz, CDCl₃): δ 7.70 (s, 2H), 7.56 (s, 2H), 7.24 (d, J=3.0 Hz, 2H), 6.78 (d, J=3.6 Hz, 2H), 2.80 (d, J=7.2 Hz, 4H), 1.67 (m, 2H), 1.32-1.27 (m, 48H), 0.89-0.85 (m, 12H).

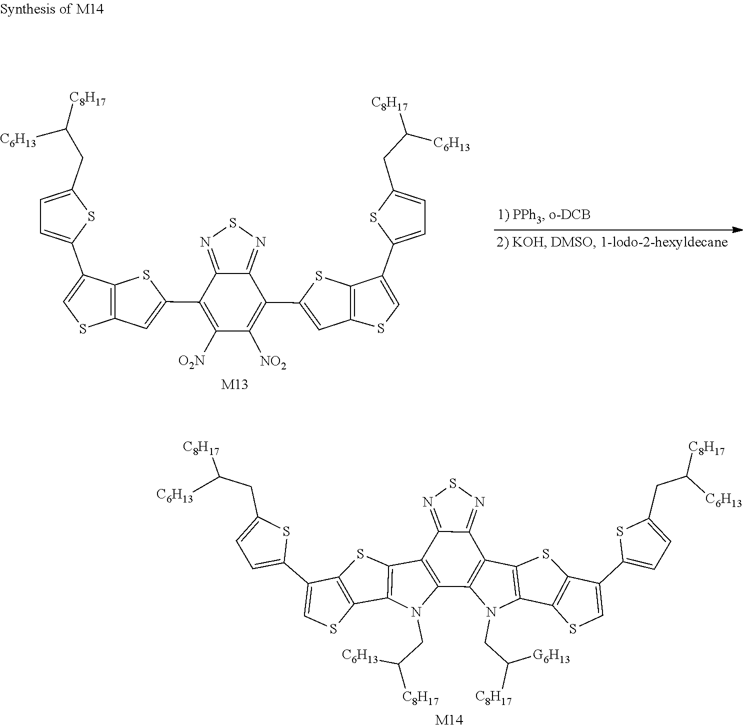

M13 (1.9 g, 1.7 mmol) and triphenylphosphine (PPh₃, 4.46 g, 17 mmol) were placed into a 250 mL three-necked reaction flask, and o-dichlorobenzene (o-DCB, 48 mL) was added in. The reaction mixture was purged with argon for 30 minutes. The temperature of the reaction mixture was heated to 180° C. and refluxed for 4.5 hours. After the reaction is completed, the three-necked flask is removed from the casserole and was cooled to room temperature. o-DCB was distilled away. The product was precipitated with tetrahydrofuran (THF, 15 mL) and methanol (MeOH, 40 mL). The solid was collected by suction filtration, and the crude product (1.39 g) was obtained. The crude product was carried directly to the next step without additional purification. The crude product (1.39 g, 1.32 mmol) and potassium hydroxide (KOH, 0.59 g, 10.6 mmol) were placed into a 100 ml three-necked flask. Dimethyl sulfoxide (DMSO, 13.9 mL) and toluene (9.5 mL) were added in and the reaction mixture was stirred at room temperature for 30 minutes. 1-iodo-2-hexyldecane (2.8 g, 7.9 mmol) was added in and the reaction mixture was heated to 80° C. for 18 hours. The temperature of the reaction mixture was cooled to room temperature. The mixture was extracted three times with ethyl acetate/water (EA/H₂O). The organic layer was collected and was dried with magnesium sulfate (MgSO₄), and the organic solvent was removed. The crude product was purified by a silica gel column, and the eluent was heptane/dichloromethane (Heptane/DCM)=3/1, and the product M14 (1.23 g, yield: 86%) was obtained as an orange-red oil. ¹H NMR (600 MHz, CDCl₃): δ 7.40 (s, 2H), 7.38 (d, J=3.6 Hz, 2H), 6.81 (d, J=3.6 Hz 2H), 4.62 (d, J=7.8 Hz, 4H), 2.81 (d, J=6.6 Hz 4H), 2.09-2.07 (m, 2H), 1.69 (m, 2H), 1.26-0.83 (m, 102H), 0.83-0.78 (m, 12H), 0.77-0.67 (m, 6H).

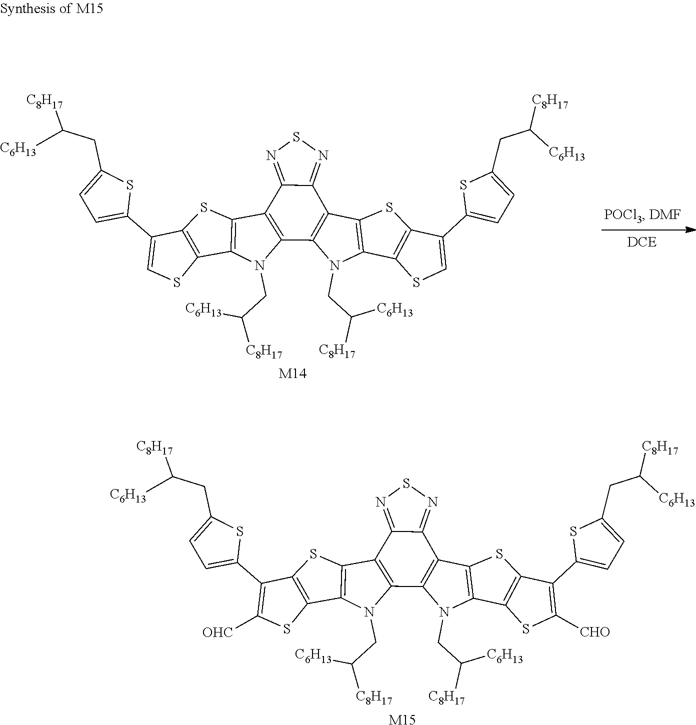

Phosphorus oxychloride (POCl₃, 0.79 mL, 4.92 mmol) and dimethylformamide (DMF, 3.17 mL, 41.0 mmol) were mixed in a 100 ml two-necked reaction flask in an ice bath, and were stirred with a magnet for 30 minutes to form Vilsmeier reagent. Prepare another 100 ml two-necked reaction flask, M14 (1.23 g, 0.82 mmol) and dichloroethane (DCE, 50 mL) were placed in and were stirred with a magnet. Then, the Vilsmeier reagent was added in, and the temperature of the reaction mixture was heated to 70° C. for 3 hours. The temperature of the reaction was cooled to room temperature, and the mixture was extracted three times with dichloromethane/water (DCM/H₂O). The organic layer was collected and was dried with magnesium sulfate (MgSO₄), and the organic solvent was removed. The crude product was purified by a silica gel column, and the eluent was heptane/chloroform=1/1, and the product M15 (1.01 g, yield: 79%) was obtained as an orange-red oil. ¹H NMR (600 MHz, CDCl₃): δ 10.26 (s, 2H), 7.44 (d, J=3.6 Hz, 2H), 6.93 (d, J=3.6 Hz, 2H), 4.65 (d, J=7.8 Hz, 4H), 2.88 (d, J=6.6 Hz, 4H), 2.05 (m, 2H), 1.75 (m, 2H), 1.43-0.86 (m, 102H), 0.80-0.78 (m, 12H), 0.70-0.07 (m, 6H).

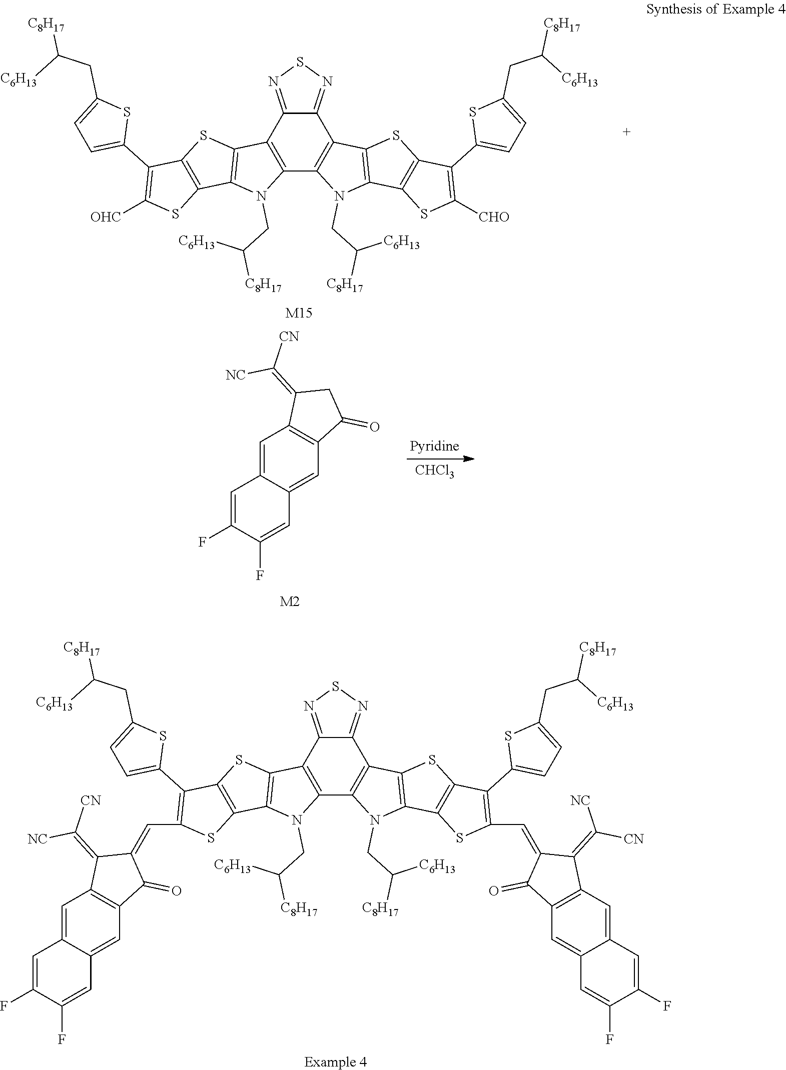

M15 (300 mg, 0.2 mmol), M2 (168 mg, 0.6 mmol) and chloroform (CF, 9 mL) were placed into a 100 mL three-necked reaction flask and was stirred with a magnet. Pyridine (0.15 mL) was added and the temperature of the reaction mixture was heated to 60° C. and reacted for 3 hours. Methanol was added to precipitate the product, and the solid was collected by suction filtration. The crude product was purified by a silica gel column, and the eluent was heptane/chloroform=1/2, and the product Example 4 (180 mg, yield: 43%) was obtained as a blue-black solid. ¹H NMR (600 MHz, CDCl₃): δ 9.24 (s, 2H), 9.10 (s, 2H), 8.33 (s, 2H), 7.87-7.84 (m, 2H), 7.83-7.80 (m, 2H), 7.41 (d, J=3.6 Hz 2H), 7.04 (d, J=3.6 Hz, 2H), 4.79 (d, J=7.2 Hz, 4H), 2.93 (d, J=6.0 Hz, 4H), 2.16-2.15 (m, 2H), 1.78 (m, 2H), 1.53-0.85 (m, 102H), 0.76-0.75 (m, 9H), 0.73-0.70 (m, 9H).

### Synthesis of Example 5

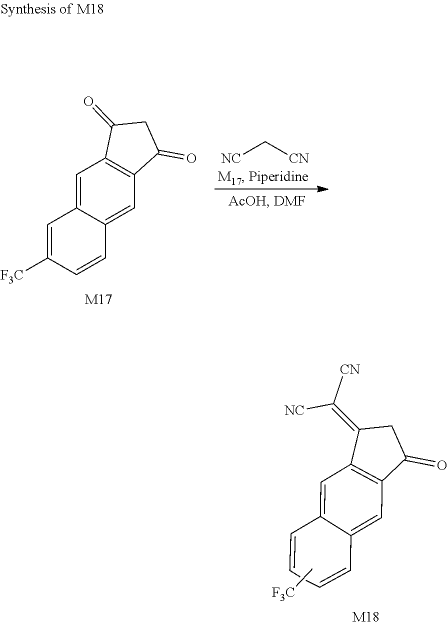

M16 (4.2 g, 15.90 mmol), M17 (2.7 g, 39.74 mmol), Piperidine (4.2 mL), acetic acid (12.6 mL) and dimethylformamide (DMF, 92.4 mL) were placed into a 100 mL two-necked reaction flask and was stirred with a magnet. The reaction mixture was reacted at room temperature for 18 hours. 1M hydrochloric acid (HCl, 252 mL) was added in and the reaction mixture was stirred at room temperature for 1 hour. The solid was collected by suction filtration and was rinsed with water and heptane. The crude product was dissolved in Ethanol (EtOH, 40 mL), and water (200 mL) was slowly added to precipitate the product. The solid was collected by suction filtration, and the product M18 (4.9 g, yield: 99%) was obtained. The ratio of the mixture was identified by ¹H NMR to be approximately 1:0.8. ¹H NMR (600 MHz, CDCl₃): δ 9.30 (s, 1H), 9.25 (s, 1H), 8.60 (s, 1H), 8.55 (s, 1H), 8.45 (s, 1H), 8.41 (s, 1H), 8.29 (d, J=10.2 Hz, 1H), 8.25 (d, J=10.2 Hz, 1H), 7.95-7.94 (m, 2H), 3.90 (s, 4H).

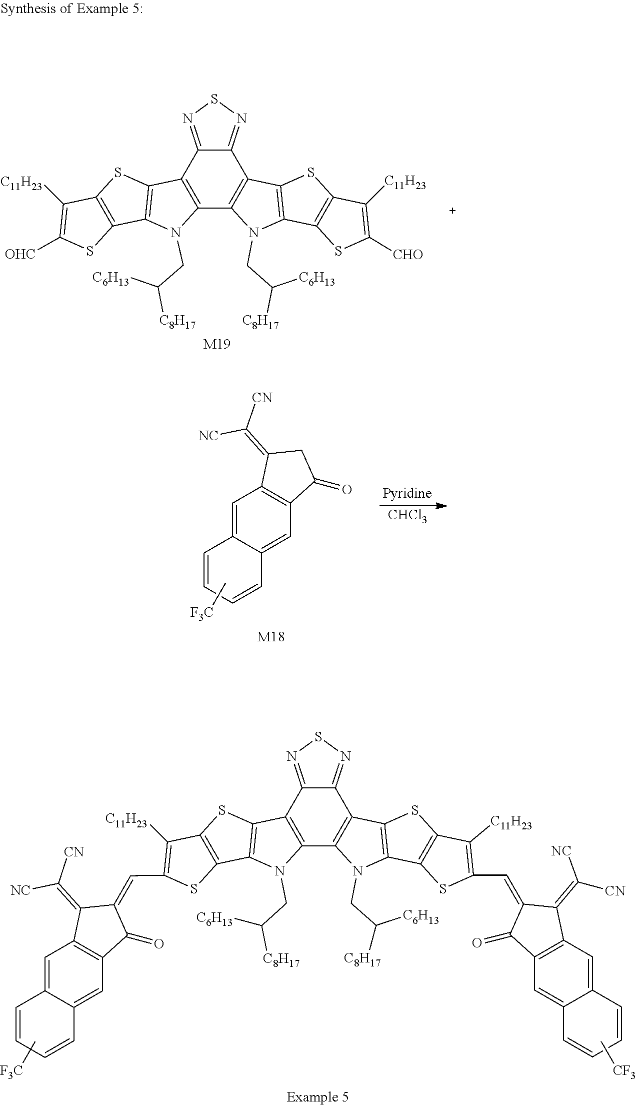

M19 (300 mg, 0.240 mmol), M18 (374 mg, 1.198 mmol) and chloroform (CF, 9 mL) were placed into a 100 mL three-necked reaction flask and was stirred with a magnet. Pyridine (0.25 mL) was added and the temperature of the reaction mixture was heated to 60° C. and reacted for 6 hours. Methanol was added to quench the reaction and precipitate the product, and the solid was collected by suction filtration. The crude product was purified by a silica gel column, and the eluent was heptane/chloroform=1/1, and the product Example 5 (340 mg, yield: 77%) was obtained as a blue-black solid. The ratio of the mixture was identified by ¹H NMR to be approximately 1:0.8. ¹H NMR (600 MHz, CDCl₃): δ 9.32 (s, 2H), 9.31 (s, 4H), 9.29 (s, 2H), 8.48 (s, 2H), 8.45 (s, 2H), 8.41 (s, 2H), 8.35 (s, 2H), 8.24 (d, J=9.0 Hz, 2H), 8.19 (d, J=9.0 Hz, 2H), 7.88-7.87 (m, 4H), 4.80 (d, J=7.8 Hz, 8H), 3.29 (t, J=7.5, 8H), 2.16 (br, 4H), 1.90-1.89 (m, 8H), 1.55-0.81 (m, 172H), 0.79-0.76 (m, 12H), 0.71-0.69 (m, 12H).

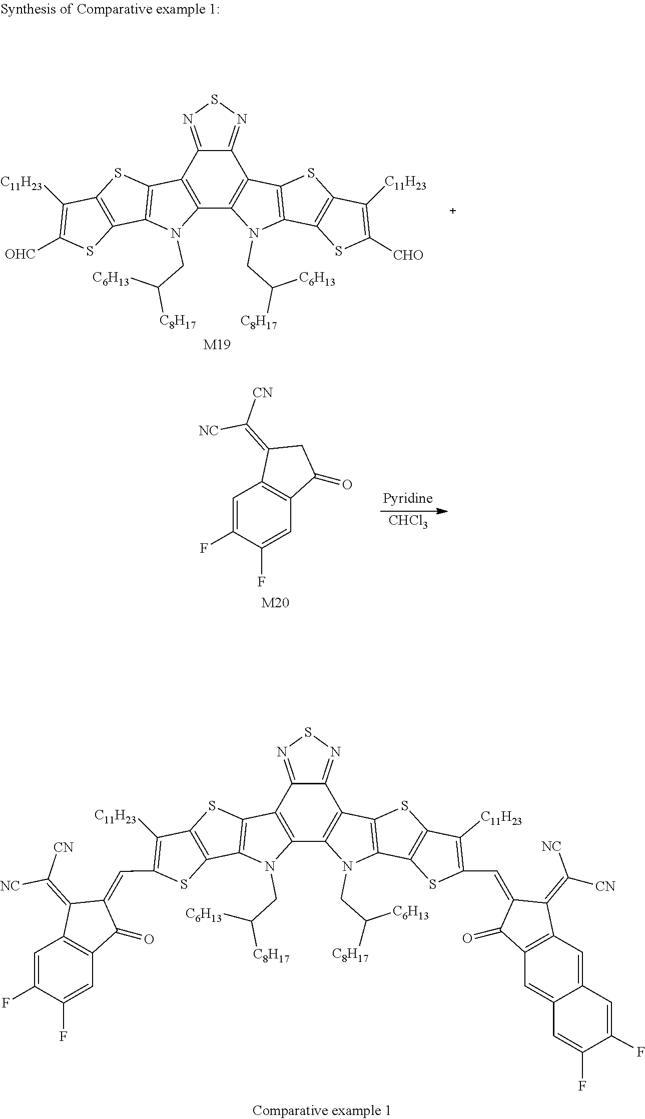

M19 (300 mg, 0.240 mmol), M20 (118 mg, 0.422 mmol) and chloroform (CF, 9.0 mL) were placed into a 100 mL three-necked reaction flask and was stirred with a magnet. Pyridine (0.15 mL) was added and the temperature of the reaction mixture was heated to 60° C. and reacted for 18 hours. Methanol was added to precipitate the product, and the solid was collected by suction filtration. The crude product was purified by a silica gel column, and the eluent was heptane/chloroform=2/1, and the product Comparative example 1 (362 mg, yield 90%) was obtained as a blue-black solid. ¹H NMR (500 MHz, CDCl₃): δ 9.16 (s, 2H), 8.57-8.57 (m, 2H), 7.71-7.68 (m, 2H), 4.76 (d, J=7.5 Hz, 4H), 3.23 (t, J=8.0 Hz, 4H), 2.13-2.10 (m, 2H), 1.91-1.85 (m, 4H), 1.56-1.49 (m, 4H), 1.39-0.77 (m, 88H), 0.69-0.67 (m, 6H).

Material testing of first acceptor material Example 1 to Example 5 and Comparative example 1. Material testing includes testing of optical properties and electrochemical properties of materials:

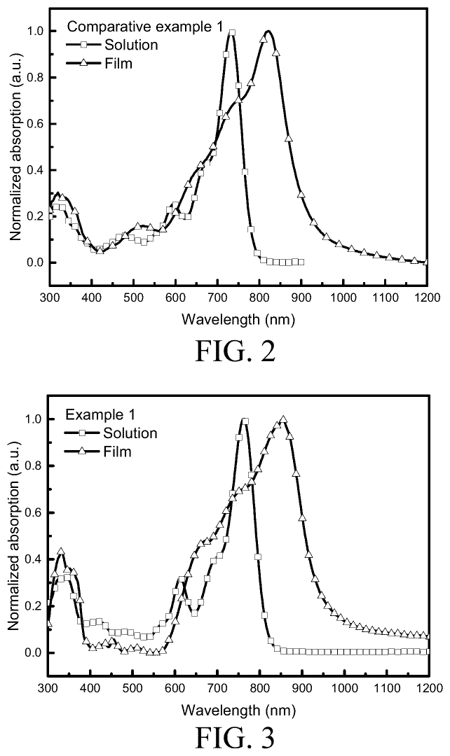

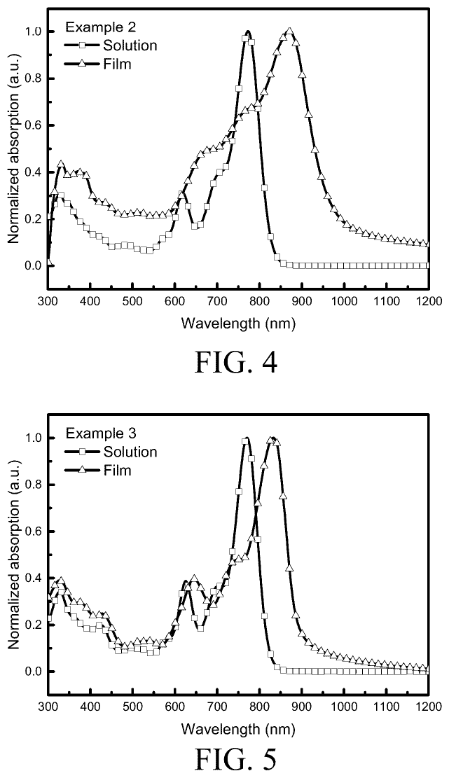

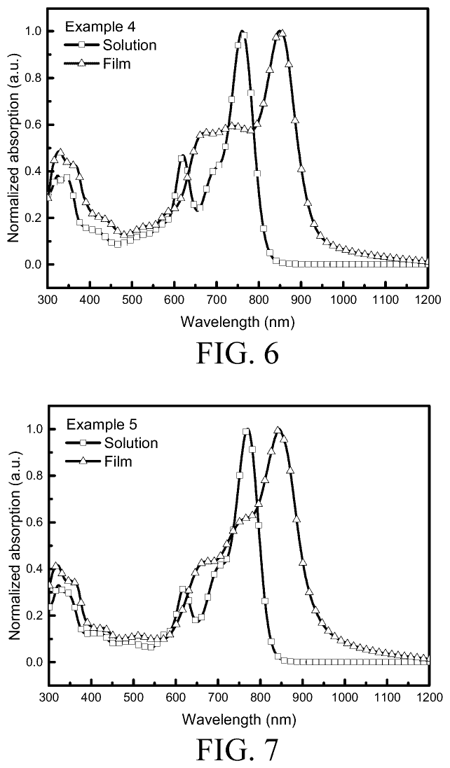

Please refer to FIG. 2 to FIG. 7 and Table 1. FIG. 2 shows an absorption spectra of a Comparative example 1 of the first acceptor material in thin film state and solution state. FIG. 3 shows an absorption spectra of an embodiment 1 of the first acceptor material of the present invention in thin film state and solution state. FIG. 4 shows an absorption spectra of an embodiment 2 of the first acceptor material of the present invention in thin film state and solution state. FIG. 5 shows an absorption spectra of an embodiment 3 of the first acceptor material of the present invention in thin film state and solution state. FIG. 6 shows an absorption spectra of an embodiment 4 of the first acceptor material of the present invention in thin film state and solution state. FIG. 7 shows an absorption spectra of an embodiment 5 of the first acceptor material of the present invention in thin film state and solution state. Table 1 shows the material test results of the first acceptor material Example 1 to Example 5 and Comparative example 1 (including the data results in FIG. 2 to FIG. 7).

Table 1. The material test results of the first acceptor material Example 1 to Example 5 and Comparative example 1

[symbol]

As shown in FIG. 2 to FIG. 7 and Table 1, the maximum absorption values of the film state of Example 1 to Example 5 are between 833 and 872 nm, and the onset absorption values are between 892 and 958 nm. From the film state absorption spectrum, it can be seen that Example 1 to Example 5 have good absorption properties at 300-900 nm, and the extinction coefficient of Example 1 to Example 5 are 1.72˜2.02×10⁵ cm⁻¹M⁻¹. In Comparative example 1, the maximum absorption value of the film state is 822 nm, and the onset absorption value is 918 nm. From the film state absorption spectrum, it can be seen that the Comparative example 1 has good absorption properties at 300-900 nm, and the extinction coefficient is 1.86×10⁵ cm⁻¹M⁻¹. The application range of the above-mentioned Example 1 to Example 5 and Comparative example 1 can be from visible light to infrared light.

Thermal stability test results for absorbance of blend material of the organic composition of the active layer:

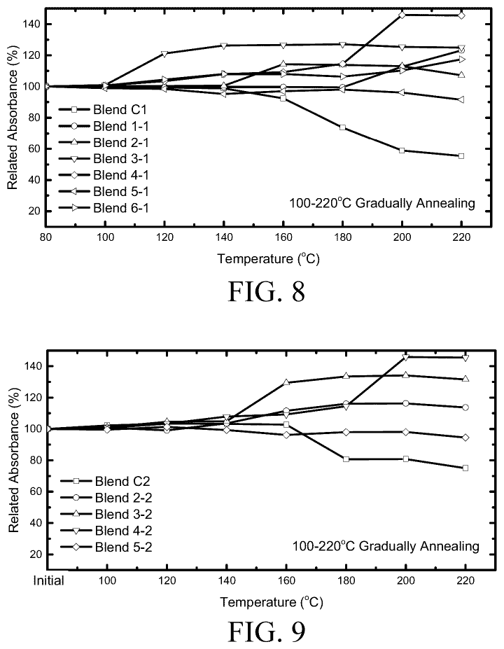

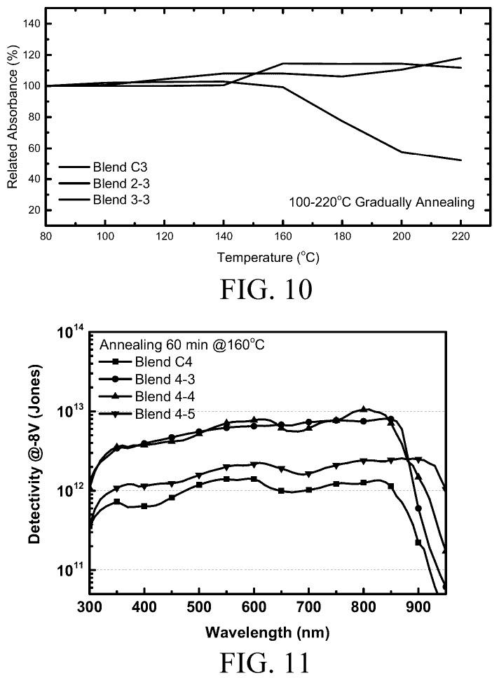

Please refer to FIG. 8 to FIG. 10 and Table 2. FIG. 8 shows thermal stability test results for absorbance of embodiments Blend 1-1 to Blend 6-1 of organic compositions of the present invention and a Comparative example Blend C1. FIG. 9 shows thermal stability test results for absorbance of embodiments Blend 2-2 to Blend 5-2 of organic compositions of the present invention and a Comparative example Blend C2. FIG. 10 shows thermal stability test results for absorbance of embodiments Blend 2-3 to Blend 3-3 of organic compositions of the present invention and a Comparative example Blend C3. Table 2 shows thermal stability test results for absorbance of blending materials shown in FIG. 8 to FIG. 10.

TABLE 2
Thermal stability test results for absorbance of blending materials shown in FIG. 8 to FIG. 10.

| | | | | Related Absorbance (%) | | | | | | | |
|---|---|---|---|---|---|---|---|---|---|---|---|
| Blend | Material | | | Initial | 100° C. | 120° C. | 140° C. | 160° C. | 180° C. | 200° C. | 220° C. |
| Blend C1 | P-type 31 | Comparative example 1 | | 100.0 | 99.8 | 99.4 | 98.8 | 92.4 | 73.8 | 58.9 | 55.4 |
| Blend 1-1 | P-type 31 | Example 1 | | 100.0 | 100.0 | 99.7 | 99.7 | 99.7 | 99.4 | 112.7 | 123.2 |
| Blend 2-1 | P-type 31 | Example 2 | | 100.0 | 100.3 | 100.4 | 100.5 | 114.3 | 113.8 | 113.0 | 107.2 |
| Blend 3-1 | P-type 31 | Example 3 | | 100.0 | 100.9 | 121.2 | 126.3 | 126.6 | 127.1 | 125.4 | 125.0 |
| Blend 4-1 | P-type 31 | Example 4 | | 100.0 | 100.8 | 103.4 | 107.9 | 109.2 | 114.5 | 145.9 | 145.5 |
| Blend 5-1 | P-type 31 | Example 5 | | 100.0 | 98.9 | 98.4 | 95.2 | 97.0 | 98.1 | 96.1 | 91.5 |
| Blend 6-1 | P-type 31 | Example 4 | Fullerene 1 | 100.0 | 100.6 | 104.4 | 108.0 | 108.0 | 106.4 | 110.2 | 117.5 |
| Blend C2 | P-type 24 | Comparative example 1 | | 100.0 | 102.2 | 103.5 | 103.2 | 102.7 | 80.6 | 80.8 | 75.0 |
| Blend 2-2 | P-type 24 | Example 2 | | 100.0 | 100.9 | 99.0 | 103.6 | 111.6 | 116.1 | 116.3 | 113.7 |
| Blend 3-2 | P-type 24 | Example 3 | | 100.0 | 101.1 | 104.6 | 104.9 | 129.5 | 133.5 | 134.1 | 131.6 |
| Blend 4-2 | P-type 24 | Example 4 | | 100.0 | 100.8 | 103.4 | 107.9 | 109.2 | 114.5 | 145.9 | 145.5 |
| Blend 5-2 | P-type 24 | Example 5 | | 100.0 | 99.5 | 101.3 | 99.3 | 96.2 | 97.9 | 98.0 | 94.6 |
| Blend C3 | P-type 9 | Comparative example 1 | | 100.0 | 102.0 | 102.6 | 102.8 | 99.2 | 77.4 | 57.7 | 52.1 |
| Blend 2-3 | P-type 9 | Example 2 | | 100.0 | 100.1 | 100.1 | 100.5 | 114.4 | 114.2 | 114.3 | 111.7 |
| Blend 3-3 | P-type 9 | Example 3 | Fullerene 1 | 100.0 | 100.6 | 104.4 | 108.0 | 108.0 | 106.1 | 110.5 | 118.0 |

Under the atmosphere, the first acceptor material Examples and the Comparative example are blended with different donor materials (p-type materials) or further added with a fullerene material of the second acceptor material to form organic compositions. Produce blending films from the aforementioned organic compositions and focus on the absorption peaks (maximum value between 750 and 950 nm) of the acceptor materials (n-type materials). As shown in FIG. 8 to FIG. 10 and Table 2, Comparative example 1 was blended with P-type 31, P-type 24 and P-type 9 to form Blend C1, C2 and C3 respectively. After Blend C1, Blend C2 and Blend C3 are gradually heated and baked, the initial absorption peak is 100%, and the relative intensity of the absorption spectrum after heating is only 52˜75%. Comparatively speaking, three different p-type materials are used in Example 1 to Example 5, and the absorbances are still maintained at 91 to 146% after gradual temperature rise and baking. In addition, ternary blending was also performed to form Blend 6-1 and Blend 3-3. The absorbances are still maintained of Blend 6-1 and Blend 3-3 still maintained at 117˜118% after gradual temperature rise and baking. It can be seen from this that Example 1 to Example 5 of the present invention still maintain good thermal stability after blending with p-type materials and fullerene materials, but Comparative example 1 cannot maintain good thermal stability.

### Preparation and Performance Testing of Organic Photodetectors of Organic Optoelectronic Devices:

A glass coated by a pre-patterned indium tin oxides (ITO) with a sheet resistance of ˜15 Ω/sq is used as a substrate. The substrate is ultrasonically oscillated in soap deionized water, deionized water, acetone, and isopropanol in sequence, and washed in each step for 15 minutes. The washed substrate is further treated with a UV-ozone cleaner for 15 minutes. The top coat of AZO (Aluminum-doped zinc oxide) solution is spin coated on the ITO substrate with a spin rate of 2000 rpm for 40 seconds, and then baked at 120° C. in air for 5 minutes to form an electron transporting layer (ETL). The active layer solution was prepared with the weight ratio of donor material: acceptor material is 1:1˜2). The concentration of the donor material was 20 mg/mL. The donor materials and the acceptor materials are as described above. After completely dissolving the active layer material, the active layer solution is filtered with PTFE filter membrane (pore size 0.45˜1.2 μm) and heated for 1 hour. Then, the active layer solution is cooled to the room temperature for spin coating, and the spin rate was used to control the film thickness in the range of 100˜800 nm. Finally, the thin film formed by the coated active layer is annealed at 100° C. for 5 minutes, and then transferred to a thermal evaporation machine. A thin layer (8 nm) of MoO₃ is deposited as a hole transporting layer (HTL) under a vacuum of 3×10⁻⁶ Torr. In this experiment, a Keithley™ 2400 source meter was used to record the dark current (J_{d}, at a bias of 0˜−8 V) in the absence of light. External quantum efficiency system was used to measure external quantum efficiency (EQE) with a range of 300-1000 nm (bias voltage 0˜−8 V), and silicon (300˜1100 nm) is used for light source calibration.

It should be noted here that, in practical applications, the first electrode preferably has good light transmittance. The first electrode is usually made of a transparent conductive material, preferably selected from one of the following conductive material groups: indium oxide, tin oxide, fluorine-doped tin oxide derivative (Florine Doped Tin Oxide, FTO), or composite metal oxides such as indium tin oxide (ITO) and indium zinc oxide (IZO). The material of the second electrode is a conductive metal, preferably silver or aluminum, more preferably silver. Suitable and preferred materials for ETL include, but are not limited to, metal oxides such as ZnOₓ, aluminum doped ZnO (AZO), TiOₓ or nanoparticles thereof, salts (such as LiF, NaF, CsF, CsCO₃), amines (such as primary amines, secondary or tertiary amines), conjugated polymer electrolytes (such as polyethyleneimine), conjugated polymers (such as poly[3-(6-trimethylammoniumhexyl)thiophene], poly(9,9)-bis(2-ethylhexyl-fluorene)-b-poly[3-(6-trimethylammoniumhexyl)thiophen e] or poly[(9,9-bis(3′-(N,N-dimethylamino))propyl)-2,7-fluorene)-alt-2,7-(9,9-dioctylfluorene)], and organic compounds such as tris(8-quinolinyl)-aluminum (III) (Alq₃), 4,7-diphenyl-1,10-phenanthroline, or a combination of one or more of the foregoing. Suitable and preferred materials for HTL include, but are not limited to metal oxides such as ZTO (Zinc Tin Oxide), MoOₓ, WOₓ, NiOₓ, SnOₓ or nanoparticles thereof, metal-containing salts, such as copper sulfide, copper thiocyanate, copper iodide, copper indium sulfide, lead sulfide, cobalt acetate, tungsten disulfide, etc., conjugated polymer electrolytes such as PEDOT:PSS, polymeric acids such as polyacrylates, conjugated polymers such as polytriarylamine (PTAA), insulating polymers such as Nafion films, polyethyleneimine or polystyrene sulfonates, organic compounds such as N,N′-diphenyl-N,N′-bis(1-naphthyl) (1,1′-biphenyl)-4,4′-diamine (NPB), N,N′-diphenyl-N,N′-(3-methylbenzene base)-1,1′-biphenyl-4,4′-diamine (TPD), or a combination of one or more of the above.

Please refer to FIG. 11 and Table 3. FIG. 11 shows detectivity test results of organic optoelectronic devices of the present invention whose active layer respectively is embodiments Blend 4-3 to Blend 4-5 and a Comparative example Blend C4. Table 3 shows the test data results of FIG. 11.

TABLE 3
The test data results of FIG. 11.

| | | | | | | EQE (%) | | D* (Jones) | | Retention D* (%) | |
|---|---|---|---|---|---|---|---|---|---|---|---|
| | | | Annealing | J_{dark} (A/cm²) | | @850 nm | | @850 nm | | @850 nm | |
| Blend | Material | | at 160° C. | −4 V | −8 V | −4 V | −8 V | −4 V | −8 V | −4 V | −8 V |
| Blend | P-type | Comparative | Initial | 3.4 × 10⁻⁹ | 7.5 × 10⁻⁹ | 63.7 | 64.9 | 1.32 × 10¹³ | 9.09 × 10¹² | 100.0 | 100.0 |
| C4 | 21 | example 1 | 30 min | 5.0 × 10⁻⁹ | 1.7 × 10⁻⁸ | 11.2 | 15.8 | 1.91 × 10¹² | 1.47 × 10¹² | 14.5 | 16.2 |
| | | | 60 min | 4.0 × 10⁻⁹ | 2.3 × 10⁻⁸ | 10.4 | 14.3 | 1.99 × 10¹² | 1.14 × 10¹² | 15.1 | 12.5 |
| Blend | P-type | Example 3 | Initial | 4.0 × 10⁻⁹ | 3.2 × 10⁻⁸ | 62.4 | 64.2 | 1.20 × 10¹³ | 4.54 × 10¹² | 100.0 | 100.0 |
| 4-3 | 21 | | 30 min | 2.7 × 10⁻⁹ | 2.5 × 10⁻⁸ | 60.3 | 63.0 | 1.41 × 10¹³ | 4.83 × 10¹² | 117.5 | 106.4 |
| | | | 60 min | 2.7 × 10⁻⁹ | 9.7 × 10⁻⁹ | 61.1 | 64.8 | 1.43 × 10¹³ | 7.98 × 10¹² | 119.2 | 175.8 |
| Blend | P-type | Example 4 | Initial | 7.9 × 10⁻⁹ | 1.7 × 10⁻⁸ | 61.9 | 64.7 | 8.43 × 10¹² | 6.01 × 10¹² | 100.0 | 100.0 |
| 4-4 | 21 | | 30 min | 2.3 × 10⁻⁹ | 4.1 × 10⁻⁹ | 39.7 | 42.3 | 1.00 × 10¹³ | 7.99 × 10¹² | 118.6 | 132.9 |
| | | | 60 min | 2.4 × 10⁻⁹ | 2.8 × 10⁻⁹ | 26.9 | 30.9 | 6.65 × 10¹² | 7.07 × 10¹² | 78.9 | 117.6 |
| Blend | P-type | Example S | Initial | 8.4 × 10⁻⁹ | 3.2 × 10⁻⁸ | 63.1 | 65.5 | 8.33 × 10¹² | 4.43 × 10¹² | 100.0 | 100.0 |
| 4-5 | 23 | | 30 min | 4.8 × 10⁻⁹ | 1.1 × 10⁻⁸ | 15.2 | 19.2 | 2.66 × 10¹² | 2.22 × 10¹² | 31.9 | 50.1 |
| | | | 60 min | 4.6 × 10⁻⁹ | 1.1 × 10⁻⁸ | 14.5 | 21.1 | 2.59 × 10¹² | 2.43 × 10¹² | 31.1 | 54.9 |

As shown in FIG. 11 and Table 3, the device tests of the active layers Blend C4, Blend 4-3, Blend 4-4 and Blend 4-5 are formed by the P-type 21 used as the donor material blending with Comparative example 1 and Example 3 to Example 5. Using 850 nm as the observation wavelength, the initial performance of the device and the performance of the device after 160° C. annealing are discussed. In terms of initial component performance, there is not much difference in the performance of four active layers. After annealing at 160° C., in the trend of dark current density at −8V, as the annealing time increases, the dark current density of Blend C4 gradually increases from 7.5×10⁻⁹ A/cm² to 2.3×10⁻⁸ A/cm². The dark current density of Blend 4-3, Blend 4-4 and Blend 4-5 all decreased to varying degrees. It needs to be understood is that the decrease in dark current density significantly improves the efficiency of organic photodetectors. It is worth noting that the EQE performance of Blend C4 at −8V declines from 64.9% to 14.3%. Blend 4-3 is almost maintained, only going from 64.2% to 64.8%. Blend 4-4 declines slightly from 64.7% to 30.9%. Although Blend 4-5 also declines from 65.5% to 21.1%, it is still higher than the 14.3% of Blend C4. Regarding the detectivity retention rate of the active layer at −8V, Blend C4 is only 15.1%, Blend 4-3 is 175.8%, Blend 4-4 is 117.6%, and Blend 4-5 is 54.9%. As a whole, the overall detectivity retention rate of the active layers of Example 3 to Example 5 at −8V is at least 3.6 times higher and can be up to 11.6 times higher than that of the active layer of the Comparative example.

Based on the above experimental results, the organic composition containing Formula I in the present invention, and the organic optoelectronic devices using it as organic photodetectors, exhibit excellent responsibility, dark current, and detectivity performance, and the devices have better stability.

With the detailed description of the above embodiments, it is hoped that the features and spirit of the present invention can be more clearly described, and the scoped of the present invention is not limited by the embodiments disclosed above. On the contrary, the intention is to cover various changes and equivalent arrangements within the scope of the patents to be applied for in the present invention.

What is claimed is:

1. An organic composition, comprising:
at least one donor material comprising at least one organic conjugated polymer or organic conjugated small molecule; and
at least one acceptor material comprising a first acceptor material, and the first acceptor material comprising Formula I: 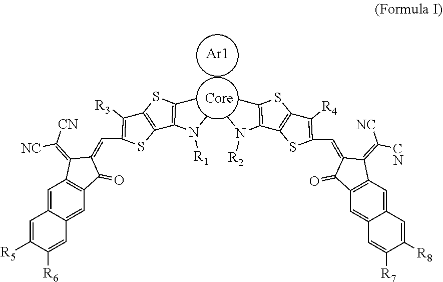
wherein core is phenyl;
Ar1 is a five-membered or six-membered heterocyclic ring comprising at least one heteroatom, and the heteroatom is independently selected from at least one of S, N, O and Se;
R₁, R₂, R₃ and R₄ are independently selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkylaryl, C1-C30 haloalkyl heteroaryl, C2-C30 esteryl aryl and C2-C30 esteryl heteroaryl; and
R₅, R₆, R₇ and R₈ are independently selected from the following groups and their derivatives consisting of C1-C30 alkyl, C1-C30 silyl, C1-C30 alkoxy, C1-C30 alkylthio, C1-C30 haloalkyl, halogen, hydrogen and cyano, wherein R₅, R₆, R₇ and R₈ are not hydrogen at the same time.

2. The organic composition of claim 1, wherein Ar1 is selected from the following structures: 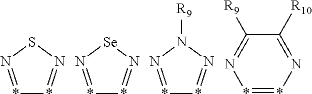
wherein R₉ and R₁₀ are independently selected from the following groups and their derivatives consisting of halogen, hydrogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl, wherein R₉ and R₁₀ are two separate groups or connected to each other by a single bond.

3. The organic composition of claim 1, wherein the donor material is further selected from at least one organic conjugated polymer.

4. The organic composition of claim 3, wherein the organic conjugated polymer is selected from the following structures: 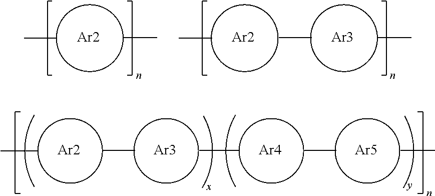
wherein, Ar2, Ar3, Ar4 and Ar5 are monocyclic or polycyclic structures containing C4-C30 ring atoms respectively;
n is the number of molecules, and n is a positive integer from 1 to 1000; and
x and y are molar fractions, where 0<x<1, 0<y<1 and x+y=1.

5. The organic composition of claim 4, wherein Ar2 and Ar4 are independently selected from the following structures: 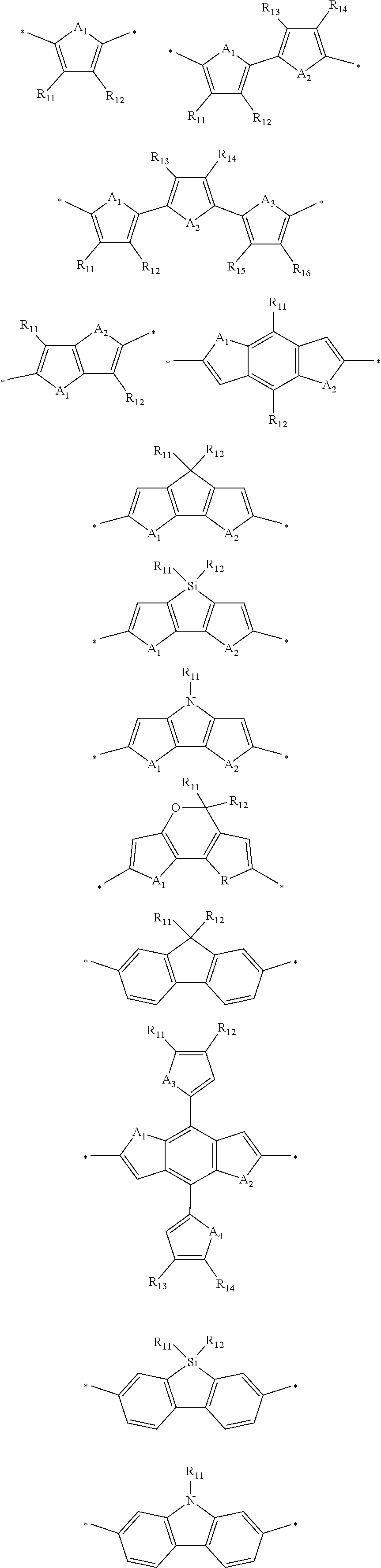
wherein, A₁, A₂, A₃ and A₄ are independently selected from the following group consisting of O, S, Se and N—R, and R is selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl; and
R₁₁, R₁₂, R₁₃, R₁₄, R₁₅ and R₁₆ are independently selected from the following groups and their derivatives consisting of hydrogen, halogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C1-C30 esteryl aryl and C2-C30 esteryl heteroaryl.

6. The organic composition of claim 4, wherein Ar3 and Ar5 are independently selected from the following structures: 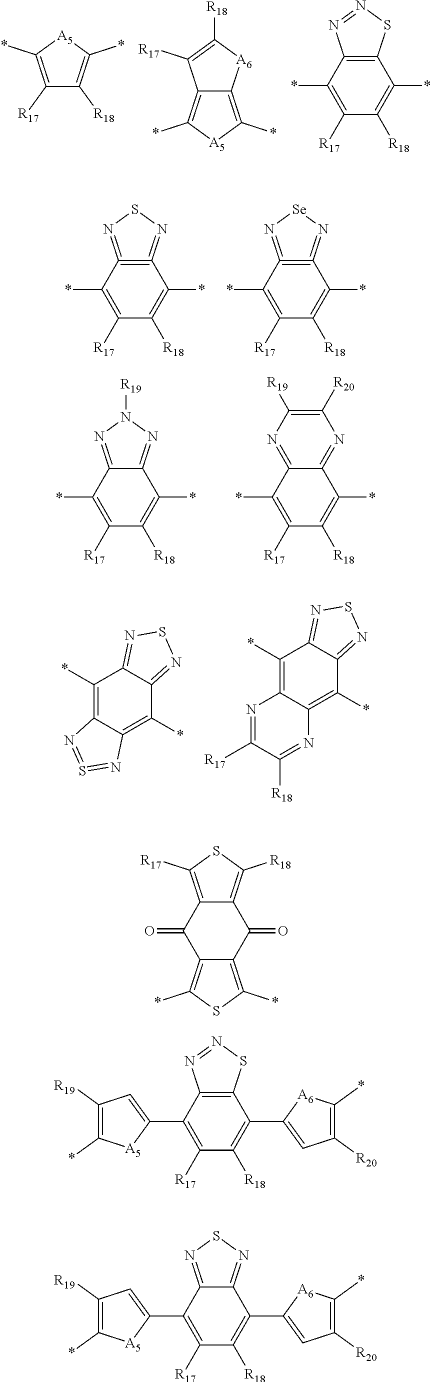 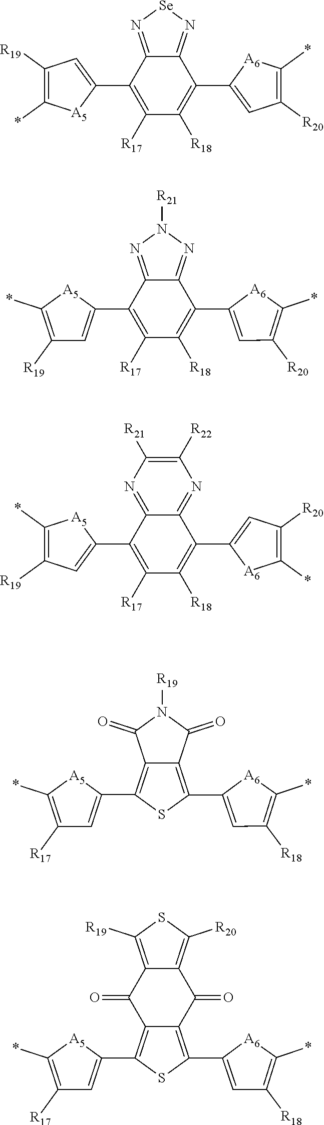 [symbol]
wherein, A₅, A₆, A₇ and A₈ are independently selected from the following group consisting of O, S, Se and N—R, and R is selected from the following groups and their derivatives consisting of C1-C30 alkyl, C1-C30 silyl, C1-C30 alkoxy, C1-C30 alkylthio, C1-C30 haloalkyl, C2-C30 ester, C1-C30 alkylaryl-, C1-C30 alkyl heteroaryl, C1-C30 silyl aryl, C1-C30 silyl heteroaryl, C1-C30 alkoxy aryl, C1-C30 alkoxy heteroaryl, C1-C30 alkylthioaryl, C1-C30 alkylthioheteroaryl, C1-C30 haloalkyl aryl, C1-C30 haloalkyl heteroaryl, C2-C30 esteryl aryl and C2-C30 esteryl heteroaryl; and
R₁₇, R₁₈, R₁₉, R₂₀, R₂₁ and R₂₂ are independently selected from the following groups and their derivatives consisting of hydrogen, halogen, cyano, C1-C30 alkyl, C1-C30 silyl, C1-C30 alkoxy, C1-C30 alkylthio, C1-C30 haloalkyl, C2-C30 ester, C1-C30 alkylaryl, C1-C30 alkyl heteroaryl, C1-C30 silyl aryl, C1-C30 silyl heteroaryl, C1-C30 alkoxyaryl, C1-C30 alkoxyheteroaryl, C1-C30 alkylthioaryl, C1-C30 alkylthioheteroaryl, C1-C30 haloalkyl aryl, C1-C30 haloalkyl heteroaryl, C1-C30 esteryl aryl and C2-C30 esteryl heteroaryl.

7. The organic composition of claim 1, wherein the acceptor material further comprises a second acceptor material, and the second acceptor material is an organic conjugated polymer or an organic conjugated small molecule.

8. The organic composition of claim 7, wherein the second acceptor material comprises a fullerene structure.

9. An organic optoelectronic device comprising:
a first electrode;
an active layer which at least comprises the organic composition of claim 1; and
a second electrode, wherein the active layer is disposed between the first electrode and the second electrode, and at least one of the first electrode and the second electrode is a transparent or semi-transparent electrode.

10. The organic optoelectronic device of claim 9, further comprising a first carrier transporting layer and a second carrier transporting layer, wherein the first carrier transporting layer is disposed between the first electrode and the active layer and the first carrier transporting layer is configured to transport carriers in the first electrode and the active layer; the second carrier transporting layer is disposed between the active layer and the second electrode and the second carrier transporting layer is configured to transport carriers in the active layer and the second electrode.
